(19) United States
(12) Patent Application Publication
(10) Pub. No.: US 2005/0233954 A1
(43) Pub. Date: Oct. 20, 2005

(54) Use of resistin to treat hematopoietic disorders

(75) Inventors: Stanley Richard Jaskunas, Indianapolis, IN (US); De-Shan Li, Indianapolis, IN (US); Ling Liu, Carmel, IN (US); Wei Zeng, Carmel, IN (US)

Correspondence Address: ELI LILLY AND COMPANY;PATENT DIVISION, P.O. BOX 6288, INDIANAPOLIS, IN, 46206-6288, US

(73) Assignee: ELI LILLY AND COMPANY

(21) Appl. No.: 10/509,767

(22) PCT Filed: Mar. 27, 2003
(86) PCT No.: PCT/US03/08456

§ 371 Date: Sep. 28, 2004

(30) Foreign Application Priority Data
Apr. 8, 2002 (US) 60370936

(51) Int. Cl. A61K 38/17

(52) U.S. Cl. 514/12

(57) ABSTRACT

The present invention relates to a method of using a mammalian gene sequence and polypeptides encoded thereby to treat mammalian hematopoietic disorders. More specifically the present invention relates to methods of using compositions comprising at least one resistin agonist, resistin polynucleotide and/or resistin polypeptide for the prevention and/or treatment of mammalian hematopoietic disorders, including, but not limited to, anemia, leukemia, and hematopoietic conditions caused by bone marrow transplantation or chemo-/radiation therapy.

### FIELD OF THE INVENTION

The present invention relates to recombinant DNA technology as applied to the field of human medicine. In particular, the invention relates to new methods of modulating mammalian myeloid cell populations and treating or preventing mammalian hematopoietic disorders comprising the administration of the protein resistin, also termed FIZZ3.

### BACKGROUND OF THE INVENTION

Hematopoiesis is an essential, lifelong process whereby highly specialized blood cells are generated from hematopoietic stem cells, including cells responsible for carbon dioxide and oxygen transport (erythrocytes), blood clotting (platelets), humoral immunity (B lymphocytes), cellular immunity (T lymphocytes), as well as cells which respond to foreign organisms and their products (granulocytes, monocytes, and macrophages). All of these cells can be functionally divided into two distinct groups termed myeloid and lymphoid. During normal human adult life, myeloid cells are produced exclusively within the bone marrow (Lichtman, M. A., Exp.Hematol. 9:391, 1981) while cells of the lymphoid lineage are produced to varying degrees in the bone marrow, spleen, thymus, and lymph nodes. Mature, functional, end cells and their immediate precursors have a limited life-span and a limited proliferative capacity and hence are not self-maintaining. Thus, these cells are continuously replaced from a pool of more primitive, proliferating, progenitor cells. Ultimately, all cells of both the myeloid and lymphoid lineage are derived from totipotent stem cells. In the normal human adult it is estimated that approximately 200 billion erythrocytes (Erslev, A. J., Hematology, McGraw-Hill, New York, 1983) and 60 billion neutrophilic leukocytes (Dancey, J. T. et al, J. Clin. Invest. 58:705, 1976) are produced everyday.

Hematopoiesis is necessarily tightly regulated. The molecules responsible for regulating various aspects of hematopoiesis can generally be divided into two groups: extracellular growth factors and intracellular factors (e.g. growth factor receptors, signaling molecules and transcriptional factors). Hematopoietic cytokines have been successfully used to treat various diseases arising from imbalances between degradation and reconstitution of blood cells or from generation of inappropriate numbers of certain blood cells. For example, recombinant erythropoietin (EPO) is a glycoprotein administered for the treatment of anemia in chronic renal failure patients, zidovudine-treated HIV-infected patients, cancer patients on chemotherapy, and patients receiving autologous transfusions. Recombinant thrombopoietin (TPO) is currently undergoing evaluation for treatment of thrombocytopenia. In spite of the availability of EPO and TPO, there remains a need to provide additional methods of altering the hematopoietic state of an individual. There is a need therefore to find novel methods in which the supply of undifferentiated or not fully differentiated cells may be increased, for example, by stimulating the proliferation of the cells without inducing differentiation. It is also desirable to modulate differentiation of undifferentiated cells in a controlled manner. Accordingly, it is an object of the present invention to provide novel methods of modulating stem cells or precursor cells of the hematopoietic system.

A recently discovered protein designated resistin, also termed FIZZ3, (amino acid sequence described in WO9858061-A1; WO9911293-A1; WO0005259-A1; and

WO9931236-A2) has been identified as a peptide hormone that belongs to a class of cysteine-rich secreted proteins designated as comprising the RELM (resistin-like molecules)/FIZZ (found in inflammatory zone) family (Steppan, et al. 2000 PNAS 98:502-506; Steppan, et al. 2000 Nature 409:307-312). Until now, resistin's function has been ill-defined (see refs. 1-20) with evidence in a mouse model demonstrating it being synthesized in white adipose tissue and functioning as an insulin antagonist (17-19) but conflicting data in humans showing it to be expressed at low levels or not at all in human adipose cells (11, 13), expressed in human mononuclear blood cells (11, 16) and having no insulin resistance function. (8, 11, 16). However, Applicants discovered and report herein that human resistin possesses the novel utility to increase the number of hematopoietic stem cells and hematopoietic precursor cells.

### SUMMARY OF THE INVENTION

The present invention provides a novel method for modulating hematopoiesis, including erythropoiesis (production of red blood cells), leukopoiesis (production of white blood cells) and/or thrombocytopoiesis (production of platelets) comprising administering a therapeutically-effective amount of a pharmaceutical composition comprising at least one resistin agonist, resistin antagonist, resistin polynucleotide, resistin polypeptide, resistin variant, and/or resistin antibody as defined herein.

The present invention further embodies a method for modulating hematopoiesis, including erythropoiesis (production of red blood cells), leukopoiesis (production of white blood cells) and/or thrombocytopoiesis (production of platelets) that comprises administering a therapeutically-effective amount of a composition comprising, alternatively, consisting of, or consisting essentially of, at least one resistin agonist, resistin polynucleotide, resistin polypeptide, resistin functional fragment or resistin variant as defined herein to a cell, tissue, organ, mammal, or patient in need of such therapy.

The invention embodies a method for increasing the number of hematopoietic progenitor cells termed CFU-GEMM (colony forming unit—granulocyte, erythroid, macrophage, megakaryocyte) cells that comprises administering a therapeutically-effective amount of a composition comprising, alternatively consisting of, or consisting essentially of, at least one resistin polypeptide, resistin polynucleotide, resistin functional fragment or resistin variant, as defined herein, to a cell, tissue, organ, mammal, or patient in need of such therapy. It is contemplated that such composition may further comprise at least one additional hematopoietic cytokine.

The present invention embodies a composition comprising, alternatively consisting of or consisting essentially of, a therapeutically effective amount of at least one resistin polypeptide, resistin polynucleotide, resistin agonist, resistin functional fragment and/or resistin variant, as defined herein. It is contemplated that such composition may further comprise a therapeutically effective amount of at least one additional hematopoietic cytokine.

### BRIEF DESCRIPTION OF THE FIGURES

FIG. 1 represents the polynucleotide sequence encoding human resistin with the start and stop codons underlined. (SEQ ID NO: 1).

FIG. 2 represents the polypeptide sequence of human resistin with the signal peptide sequence underlined. The “*” represents an optional stop codon. (SEQ ID NO: 2)

FIG. 3 represents the polypeptide sequence of the mature form of human resistin lacking a signal peptide sequence. The “*” represents an optional stop codon. (SEQ ID NO: 3).

FIG. 4 represents the sequence alignment of human resistin with resistin-like proteins disclosed in various patent specifications. The “Xaa's” in the line defining a 100% consensus sequence refers to either R or L at the “Xaa⁴⁸” position, G or E at the “Xaa⁴⁹” position, C or S at the “Xaa⁵⁰” position and Q or E at the “Xaa⁵¹” position.

FIG. 5 represents the sequence alignment of rat, mouse and human resistin proteins (SEQ ID Nos. 13, 14 and 2, respectively.

FIG. 6 represents the hematopoietic progenitor cell differentiation scheme.

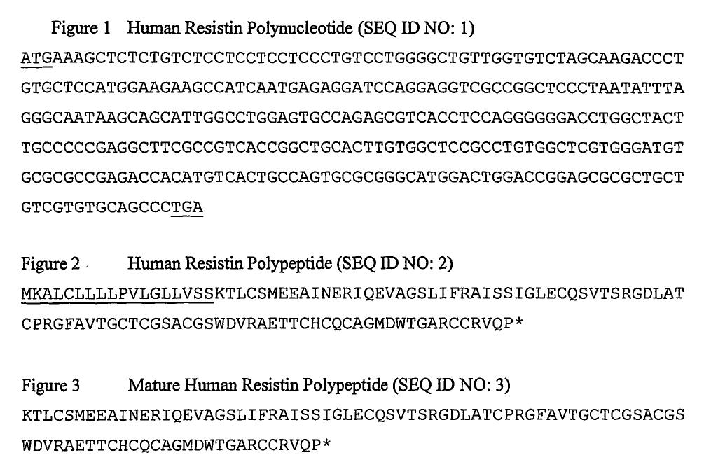

### DETAILED DESCRIPTION OF THE INVENTION

The invention is not limited to the particular embodiments described below, as variations of the particular embodiments may be made and still fall within the scope of the appended claims. The terminology used herein is for the purpose of describing particular embodiments, and is not intended to be limiting. Instead, the scope of the present invention will be established by the appended claims.

In this specification and the appended claims, the singular forms “a”, “an”, and “the” include plural reference unless the context clearly dictates otherwise.

Definitions

The term “resistin agonist” is used in the broadest sense and includes any molecule that induces or increases the expression, stability, and/or biological activity of any resistin polynucleotide or resistin polypeptide. resistin agonists may include, for example, small molecules, resistin polypeptides, resistin polynucleotides and resistin fragments as defined herein. Suitable resistin agonists include, but are not limited to, resistin polypeptides, as well as fragments thereof, resistin variants, and small organic molecules. Methods for identifying resistin agonists may comprise contacting a resistin polypeptide with a candidate resistin agonist molecule and measuring a detectable change in one or more biological activities normally associated with the resistin polypeptide, e.g., increase in number of CFU-GEMM.

The term “fragment” or “fragment thereof” in reference to a resistin gene or cDNA sequence or complementary sequence, refers to a segment of a resistin nucleic acid that comprises at least 15 or more nucleotides, more preferably at least 30, 40 or 45 or more nucleotides, that are contiguous in the native nucleic acid molecule as shown in SEQ ID NO: 1 or its complementary sequence.

The term “fragment” or “fragment thereof” in reference to a resistin polypeptide sequence, refers to a segment of a resistin polypeptide that comprises 15 or more amino acids that are contiguous in the native polypeptide as shown in SEQ ID NOs: 2, 3, 4, 5, 6 or 7.

The term “functional” in reference to a resistin polynucleotide, resistin polypeptide and/or resistin variant is intended to mean that the particular molecule exhibits a biological activity, in vivo or in vitro, that is similar or identical to a biological activity attributable to resistin polypeptides as disclosed herein (e.g., the ability to induce, or encode a polypeptide that induces the growth or differentiation of hematopoietic progenitor cells).

“Functional fragment” as used herein, refers to an isolated segment of a resistin polypeptide or resistin polynucleotide or complement thereof, that comprises a functionally distinct region such as an active site on an enzyme, a binding site for a resistin agonist or a resistin polypeptide. Functional fragments may be produced by means readily known to those in the art including, but not limited to, recombinant DNA methodologies or enzymatic/proteolytic digestions.

The term “hematocrit” refers to a measurement of the ratio of the volume of red blood cells to the volume of whole blood cells as determined by any instrument used in determining the relative amounts of plasma and corpuscles in blood. It is understood that hematocrit targets will vary from one individual to another such that physician discretion may be appropriate in determining an actual target hematocrit for any given patient.

The term “resistin” refers to a nucleic acid, gene, cDNA (as shown in SEQ ID NO: 1), fragments thereof and sequence complementary to SEQ ID NO: 1 as well as to any polypeptide sequence (as shown in SEQ ID NO: 2-7) encoded thereby. The term “resistin” without further limitation also refers to both the native resistin polypeptide (SEQ ID NO: 2) as well as the mature form of the resistin polypeptide (i.e., SEQ ID NO: 3) and the polynucleotide encoding such polypeptide. If not stated otherwise, the term “resistin polypeptide” encompasses the full-length and functional fragments of the resistin polypeptide as shown in SEQ ID NO: 2-7 as well as, secreted, mature, fused, variant, alternatively spliced, and allelic forms thereof (e.g., as shown in SEQ ID NO: 3).

The term “resistin composition” is intended to refer to a composition of matter comprising at least one resistin agonist, resistin polynucleotide, resistin polypeptide, resistin fragment, resistin functional fragment, resistin variant and/or resistin fusion protein as defined herein.

The term “resistin variant” in reference to a resistin polynucleotide is intended to refer to a polynucleotide sequence that encodes a polypeptide of SEQ ID NO: 2-7 or a polypeptide with at least 95%, more preferably at least 96%, 97%, 98% or 99% sequence identity to a sequence shown in SEQ ID NO: 2-7, but which differs from the polynucleotide sequence shown in SEQ ID NO: 1 due, e.g., to the variance of the amino acids from those of wild type or the degeneracy of the genetic code. The term “resistin variant” in reference to a resistin polypeptide is intended to refer to a polypeptide with a sequence at least 95%, more preferably at least 96%, 97%, 98% or 99% sequence identity to a sequence shown in SEQ ID NOS: 2-7.

The term “hematopoietic cytokine” as used herein refers to any protein such as, for example, a cytokine or growth factor or hormone that functions to induce proliferation and/or differentiation of hematopoietic progenitor cells.

The term “isolated” when used in relation to a nucleic acid or protein, refers to a nucleic acid sequence or protein that is identified and separated from at least one contaminant with which it is ordinarily associated in its natural source. Isolated nucleic acid or protein is present in a form or setting that is different from that in which it is found in nature. In contrast, non-isolated nucleic acids or proteins are found in the state they exist in nature.

As used herein, the term “purified” or “to purify” means the result of any process which removes some contaminants from the component of interest, such as a protein or nucleic acid. The percent of a purified component is thereby increased in the sample.

The term “mature protein” or “mature polypeptide” as used herein refers to the form(s) of a protein produced by expression in a mammalian cell. It is generally hypothesized that once export of a growing protein chain across the rough endoplasmic reticulum has been initiated, proteins secreted by mammalian cells have a signal peptide (SP) sequence that is cleaved from the complete polypeptide to produce a “mature” form of the protein. Oftentimes, cleavage of a secreted protein is not uniform and may result in more than one species of mature protein. The cleavage site of a secreted protein is determined by the primary amino acid sequence of the complete protein and generally cannot be predicted with complete accuracy. Methods for predicting whether a protein has a SP sequence, as well as the cleavage point for that sequence, are available. A cleavage point may exist within the N-terminal domain of resistin between amino acid 15 and amino acid 25, most preferably after amino acid 18 of the sequence shown in SEQ ID NOs: 2,4-7. As one of ordinary skill would appreciate, cleavage sites sometimes vary from organism to organism and cannot be predicted with absolute certainty. Optimally, cleavage sites for a secreted protein are determined experimentally by amino-terminal sequencing of the one or more species of mature proteins found within a purified preparation of the protein.

The term “treatment” or “treating” as used herein describes the management and care of a patient for the purpose of combating or preventing a hematopoietic disease, condition, or disorder and includes the administration of a resistin agonist, resistin polynucleotide, resistin polypeptide, resistin fragment, resistin functional fragment, resistin variant, and/or a resistin composition to prevent or delay the onset of the symptoms or complications, alleviate or reduce the severity of the symptoms or complications, or eliminate or reduce the severity of the disease, condition, or disorder.

### DETAILED DESCRIPTION OF THE INVENTION

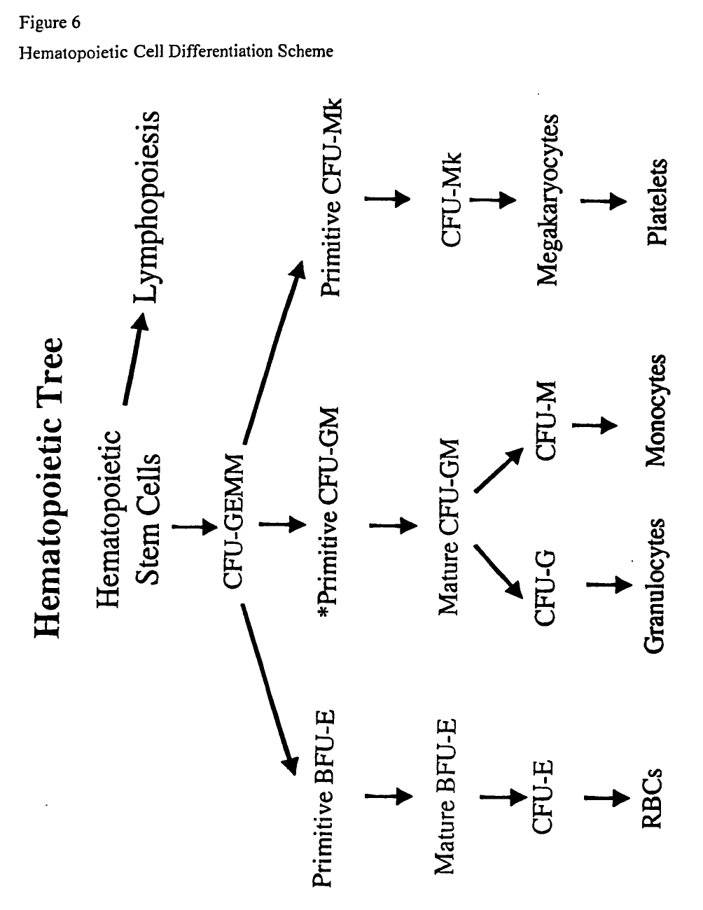

The present invention demonstrates that resistin activity is closely associated with modulating proliferation and/or differentiation of cells of the myeloid branch of the hematopoietic system. For example, Applicants have shown that a resistin polypeptide, in combination with at least one other hematopoietic cytokine, when administered to human CD34+cells, significantly increases the number of CFU-GEMM cells and ultimately may increase the number of red blood cells, platelets, granulocytes and monocytes. (See Example 5 herein). It is futher contemplated that resistin may have this effect when administered alone to a patient in need thereof. Addition of resistin+erythropoietin (EPO)+IL-3 to human bone marrow cells (CD34+) significantly increased the number of CFU-GEMM formed when compared to addition of EPO+IL-3 without resistin. Addition of resistin to CD34+cells also increased the number of primitive BFU-E cells when also added with EPO and SCF or EPO+SCF+IL-3 compared to the number of BFU-E when resistin was not added. For the position of CFU-GEMM and BFU-E in the hematopoietic progenitor cell scheme see FIG. 6. CFU-GEMM cells are pluripotent prescursor cells that can give rise to differentiated cells of the myeloid lineage such as, e.g., red blood cells, platelets, neutrophils, granulocytes, basophil/mast cells, eosinophils and monocyte/macrophages. These colonies also contained increased numbers of megakaryocyte cells, further supporting the observation that resistin stimulates a hematopoietic progenitor cell in common for both erytirocyte and megakaryocyte pathways.

The catagorization of myeloid lineage cells descendant from progenitor CFU-GEMM's is generally described as shown in FIG. 6. CFU-GEMM is a colony-forming unit-granulocyte, erythroid, macrophage, megakaryocyte; BFU-E is a Burst-forming unit-erythroid; RBC is a red blood cell; CFU-GM is a colony-forming unit-granulocyte, macrophage; CFU-G is a colony-forming unit-granulocyte; CFU-M is a colony-forming unit-monocyte; and CFU-Mk is a colony-forming unit-megakaryocyte.

Anemia occurs when the body is unable to produce enough red blood cells resulting in a decrease in the oxygen carrying capacity of the blood. Anemia commonly occurs in patients with cell proliferation diseases, e.g., cancer and as a side effect of the treatment of such diseases. Thrombocytopenia confers a risk of bleeding resulting from the presence of abnormally low levels of platelets in the circulating blood and is a common side effect of chemotherapy. Cancer patients may also experience thrombocytopenia from other medications or as a consequence of their underlying cancer. Endogenous cytokines such as EPO, TPO, SCF (among others) are typically upregulated following chemotherapy or radiation therapy as would be needed, for example, by a cancer patient receiving treatment for anemia or thrombocytopenia. Thus, resistin polypeptides and variants thereof have the potential to act as a monotherapy for enhancing the recovery of platelets and/or erythrocytes following such treatment by increasing the number and/or cycling status of hematopoietic progenitor cells, particularly CFU-GEMM cells and their descendents.

Cytokines currently approved to treat myelosuppression (a reduction in the ability of the bone marrow to produce blood cells) include G-CSF, EPO, IL-11 and GM-CSF; however, only EPO and G-CSF are widely used to treat anemia and neutropenia respectively. Therefore, the present invention provides methods of treating or preventing hematopoietic disorders in mammals, preferably humans, comprising the administration of a therapeutically effective amount of a pharmaceutical composition comprising at least one resistin agonist, resistin polynucleotide, resistin functional fragment, resistin variant, and/or resistin polypeptide of the invention.

Therapeutic Uses

The invention provides for the treatment and/or prevention of diseases, disorders, syndromes, and/or conditions in which modulation of the numbers of one or more hematopoietic cell types is desired (e.g., diseases, disorders, syndromes, and/or conditions associated with one or more hematopoietic cell deficiencies) by administration of a composition comprising a therapeutic compound (termed herein “Therapeutic”) of the invention to modulate (e.g., by enhancing or stimulating) hematopoiesis, erythropoiesis, leukopoiesis, thrombocytopoiesis, production of neutrophils, granulocytes, and/or platelets by, e.g., stimulating the proliferation and/or differentiation of progenitors of such cells; or by administration of hematopoietic cells, the production of which has been induced in vitro or ex vivo by contacting the cells with a Therapeutic of the invention. Such “Therapeutics” include but are not limited to: resistin, and/or therapeutically or prophylactically effective resistin peptides or functional fragments, e.g., such as those resistin peptides which prevent or treat hematopoietic deficiencies (e.g., as demonstrated in in vitro and in vivo assays described herein or otherwise known in the art) as well as modifications, derivatives and analogs thereof and nucleic acids encoding resistin and therapeutically and prophylactically effective resistin peptides, and derivatives and analogs thereof or resistin polynucleotide.

In one embodiment, a Therapeutic of the invention is a polypeptide comprising an amino acid sequence of a therapeutically and/or prophylactically effective portion of resistin (SEQ ID NOs: 2-7) In another embodiment, the Therapeutic of the invention is a polypeptide having a sequence as shown in SEQ ID NO: 2 or 3. In other embodiments, the Therapeutic of the invention is a monomer, a dimer, or a multimer, e.g., such as a trimer or a tetramer (according to, e.g., SDS-PAGE (under non-reducing conditions) or electrospray mass) of human resistin. The Therapeutic is preferably in dimer formation. In another embodiment, a Therapeutic of the invention is a combination of any of the above resistin forms, e.g., such as a complex of resistin monomers, resistin dimers, and/or resistin higher-order forms (e.g., such as a tetramer).

In one embodiment of the invention, the Therapeutic is administered directly to a subject suffering from a disease, disorder, syndrome, and/or condition amenable to treatment by modulating (e.g., increasing) production of one or more hematopoietic cell types (e.g., a disease, disorder, syndrome, and/or condition associated with a hematopoietic cell deficiency). In an ex vivo embodiment of the invention, hematopoietic cells, preferably stem and/or progenitor cells, are obtained, then contacted with a Therapeutic of the invention in vitro to induce proliferation of the cells, optionally contacted with an additional cytokine, and subsequently administered back to a subject, preferably the same subject from whom the cells were originally taken, suffering from a disease, disorder, syndrome, and/or condition associated with a hematopoietic cell deficiency. In a preferred embodiment, autologous hematopoietic cells (obtained from a subject or its identical twin) are reintroduced into the subject after in vitro expansion. In this embodiment, gene therapy methods can be optionally performed by introducing a resistin nucleic acid of interest, e.g., containing a polynucleotide of the invention which provides a desired effect in a subject, into the hematopoietic cells, before or after expansion of the cells by contact with a Therapeutic. Hematopoietic cell subpopulations can be isolated for use, before or after expansion in vitro. For example, blood cells can be isolated and expanded, and optionally also differentiated, in vitro, followed by introduction of all or a portion of the cells (e.g, purified platelets, red blood cells, lymphocytes, etc.) into a subject.

In general, five broad categories of diseases, disorders, syndromes, and/or conditions that can be treated by methods of the invention include without limitation: (1) diseases resulting from a failure or dysfunction of normal blood cell production and maturation (i.e., aplastic anemia, cytopenias and hypoproliferative stem cell disorders); (2) neoplastic, malignant diseases in the hematopoietic organs (e.g., leukemia and lymphomas); (3) subjects with a broad spectrum of malignant solid tumors of non-hematopoietic origin. Induction of hematopoietic cell proliferation or administration of replacement hematopoietic cells in these subjects serves as a bone marrow rescue procedure, which is provided following chemotherapy or irradiation of the malignant tumor; (4) autoimmune conditions, where the hematopoietic cells serve as a source of replacement of an abnormal immune system; and (5) genetic disorders that can be treated by infusion of hematopoietic stem cells, preferably syngeneic, which prior to transplantation have undergone gene therapy. Non-limiting examples of diseases, disorders, syndromes, and/or conditions that can be treated by induction of hematopoietic cell production in vivo or by administration of hematopoietic cells expanded in vitro (i.e., ex vivo) include without limit, for example:

Diseases resulting from a failure or dysfunction of normal blood cell production and maturation: (1) Hyperproliferative stem cell disorders, aplastic anemia, pancytopenia, agranulocytosis, thrombocytopenia, red cell aplasia, Blackfan-Diamond syndrome due to drugs, radiation, or infection idiopathic; (2) Hematopoietic malignancies: acute lymphoblastic (lymphocytic) leukemia, chronic lymphocytic leukemia, acute myelogenous leukemia, chronic myelogenous leukemia, acute malignant myelosclerosis, multiple myeloma, polycythemia vera, agnogenic myelometaplasia, Waldenstrom's macroglobulinemia, Hodgkin's lymphoma, non-Hodgkin's lymphoma; (3) Immunosuppression in subjects with malignant, solid tumors malignant melanoma, carcinoma of the stomach, ovarian carcinoma, breast carcinoma, small cell lung carcinoma, retinoblastoma, testicular carcinoma, glioblastoma, rhabdomyosarcoma, neuroblastoma, Ewing's sarcoma lymphoma; (4) Autoimmune diseases: Rheumatoid arthritis, diabetes type I, chronic hepatitis, multiple sclerosis, systemic lupus erythematosus; (5) Genetic (congenital) disorders: Anemias, familial aplastic Fanconi's syndrome, Bloom's syndrome, pure red cell aplasia (PRCA), dyskeratosis congenital, Blackfan-Diamond syndrome, congenital dyserythropoietic syndromes I-IV, Chwachmann-Diamond syndrome, dihydrofolate reductase deficiencies, formamino transferase deficiency, Lesch-Nyhan syndrome, congenital spherocytosis, congenital elliptocytosis, congenital stomatocytosis, congenital Rh null disease, paroxysmal nocturnal hemoglobinuria, G6PD (glucose-6-phosphate dehydrogenase) variants 1,2,3; pyruvate kinase deficiency, congenital erythropoietin sensitivity deficiency, sickle cell disease and trait, thalassemia alpha, beta, gamma; met-hemoglobinemia, congenital disorders of immunity, severe combined immunodeficiency disease (SCID); (6) Bare Lymphocyte Syndrome: Ionophore-responsive combined immunodeficiency, combined immunodeficiency with a capping abnormality nucleoside phosphorylase deficiency, granulocyte actin deficiency, infantile agranulocytosis Gaucher's disease, adenosine deaminase deficiency, Kostmann's syndrome reticular dysgenesis, congenital leukocyte dysfunction syndromes; (7) Inadequate Platelet Production: Aplastic anemia, refractory anemias, leukemia, preleukemial myelodysplastic syndromes, megaloblastic anemia, chemotherapy or radiation therapy, and existing platelet deficiency or an expected platelet deficiency (e.g., because of planned surgery including, but not limited to, organ/bone marrow transplantations); (8) Abnormal Platelet Function:Glanzmann's thrombasthenia, acute/chronic leukemia, myeloproliferative disorders, uremia, platelet storage pool disease, Von Willebrand disease, and postoperative cardiovascular dysfunction, and (9) Blood Coagulation Disorders: Afibrinogenemia; or wounds of any origin; and (10) Others: wound healing, Osteopetrosis, myelosclerosis, acquired, hemolytic, anemias, acquired, immunodeficiencies, infectious disorders causing primary or secondary immunodeficiencies, bacterial, infections, (e.g., Brucellosis, Listeriosis, tuberculosis, leprosy), parasitic infections, (e.g., malaria, Leishmaniasis), fungal infections, disorders involving disproportions in lymphoid cell sets and impaired immune functions due to aging, Kostmann's agranulocytosis, chronic granulomatous disease, Chediak-Higachi syndrome, neutrophil actin deficiency, neutrophil membrane GP-180 deficiency, metabolic storage diseases, mucopolysaccharidoses, mucolipidoses, miscellaneous disorders involving immune mechanisms, Wiskott-Aldrich Syndrome, and alpha 1-antitrypsin deficiency.

In one aspect, a Therapeutic of the invention is used to treat a disease resulting from a failure or dysfunction of normal blood cell production and maturation, such as an aplastic anemia, a cytopenia or a hypoproliferative stem cell disorder. These disorders entail failure of stem cells in bone marrow to provide normal numbers of functional blood cells. The aplastic anemias result from the failure of stem cells to give rise to the intermediate and mature forms of red cells, white cells, and platelets. While red cell production is usually most seriously affected, a marked decrease in production of other mature blood cell elements is also seen as some anemias specifically affect production of white cells and/or platelets.

The large majority of these anemias are acquired during adult life, and do not have any apparent genetic predisposition. About half of these acquired anemias arise in the absence of any obvious causative factor such as exposure to poisons, drugs or disease processes that impair stem cell function; these are termed idiopathic aplastic anemias. The remaining cases are associated with exposure to an extremely diverse array of chemicals and drugs and also occur as the consequence of viral infections, such as HIV infection, and after pregnancy. Other specific types of aplastic anemia are termed agranulocytosis or thrombocytopenia to indicate that the major deficiency lies in particular white cells or in platelet production, respectively. These non-red blood cell deficiencies are also often associated with HIV infection. Also significantly associated with HIV infection is a severe platelet deficiency. Additionally, agranulocytosis may be associated with autoimmune syndromes such as systemic lupus erythematosus (SLE) or with other infections, such as neonatal rubella.

In addition, immune deficiencies which are the primary or secondary result of infection by pathogenic microorganisms can be treated by administration of a Therapeutic of the invention. For example, immune deficiencies caused by microorganisms which are intracellular pathogens of hematopoietic cells, can be treated by the provision of new hematopoietic cells. These new hematopoietic cells can be generated by contacting hematopoietic stem and/or progenitor cells in vitro or in vivo with a Therapeutic of the invention to cause proliferation of the cells. Microorganism infections causing immune deficiencies which may be treated according to this embodiment of the invention include but are not limited to gram negative Bacilli such as Brucella or Listeria, the mycobacterium which are the etiological agents of tuberculosis or of Hansen's disease (leprosy), parasites such as Plasmodium (the etiological agents of malaria) or Leishmania, and fungi (such as those that cause pneumonia and other lethal infections secondary to immunodeficiencies).

In a preferred embodiment of the invention, a Therapeutic of the invention is administered for the treatment of a cytopenia associated with HIV infection. The hematopoietic deficiencies associated with HIV infection include reduction in CD4+ T-cells and other lymphocytes, red blood cells, platelets, specifically ITP, and neutrophils. Such a disorder is treated by contacting hematopoietic stem and/or progenitor cells in vitro with a Therapeutic of the invention and then infusing the resulting hematopoietic cells into the subject in need of treatment. In another preferred embodiment, the disorder is treated by direct administration of a Therapeutic of the invention to the subject in need of treatment. Assays for determining the efficacy of particular Therapeutics for treatment of hematopoietic deficiencies associated with HIV infection are known in the art or described herein.

Treatment of Malignancies

Hyperproliferative malignant stem cell disorders as well as non-hematopoietic malignancies can be treated with chemotherapy or radiation therapy along with rescue of hematopoietic cells by direct administration of a Therapeutic of the invention or by administration of hematopoietic cells induced to proliferate by contacting the cells with a Therapeutic of the invention. The conditions that can be treated according to the invention include without limitation, leukemias and solid tumors.

These malignancies are currently treated by chemotherapy and, when feasible, allogeneic bone marrow transplantation. Induction of hematopoietic cell proliferation in vivo or provision of autologous hematopoietic stem and progenitor cells expanded by administration of a Therapeutic in vitro permits hematopoietic reconstitution of subjects lacking suitable allogeneic donors and eliminates the risks of graft versus host disease arising from allogeneic marrow transplantation. Thus, in a specific embodiment, a Therapeutic is used to induce proliferation in hematopoietic cells that are subsequently administered to a subject who has undergone chemotherapy or radiation therapy for treatment of cancer or an immunological disorder. In another embodiment, a Therapeutic is directly administered to a subject who has undergone chemotherapy or radiation therapy for treatment of cancer or an immunological disorder.

Autoimmune Disorders

Many chronic inflammatory and degenerative diseases are characterized by a continuous immune reaction against the body's own tissues. Such autoimmune disorders include but are not limited to rheumatoid arthritis and other inflammatory osteopathies, diabetes type I, chronic hepatitis, multiple sclerosis, and systemic lupus erythomatosus. Autoimmune disorders are often treated by lymphoid irradiation. Administration of a Therapeutic of the invention or of cells produced upon exposure to a Therapeutic in vitro can be useful to repopulate the hematopoietic system after radiotherapy.

Anti-inflammatory drugs such as steroids retard the inflammatory cells which are activated by autoreactive T-cells, but do not prevent T-cells which recognize self-proteins from activating new inflammatory cells. A more direct approach to treating autoimmune diseases depends on eradication of T-cells by irradiation of the lymphoid tissues, and relying on stem cells from the un-irradiated bone marrow to repopulate a subject's hematopoietic system. The rationale is that the formation of new populations of mature T-cells from bone marrow stem cells may result in absence of T-cells that have reactivity to self-specific antigens. This procedure, called total lymphoid irradiation (TLI), has been used to treat intractable rheumatoid arthritis (Strober, et al., 1985 Annals of Internal Medicine 102:441). These clinical trials showed that in the majority of otherwise intractable cases, joint disease was significantly alleviated for at least 2-3 years. However, the major drawback to such treatment is failure of stem cells in the bone marrow of these elderly subjects to efficiently repopulate the hematopoietic system, resulting in infections and bleeding disorders. Analogous studies have been made of the effects of TLI as an alternative to cytotoxic drugs for treatment of SLE (Strober, S., et al., 1985, Ann. Internal Med. 102:450). Studies of the use of TLI to treat intractable SLE have also shown that this treatment alleviates disease activity, but is severely limited by failure of bone marrow stem cells to rapidly and efficiently repopulate the hematopoietic system after irradiation.

Thus, a Therapeutic of the invention can be administered to promote proliferation of the remaining hematopoietic cells to increase the success of TLI therapy. Additionally, hematopoietic stem and progenitor cells can be isolated from the subject before treatment, induced to proliferate in vitro and then introduced into the subject after TLI treatment to repopulate the hematopoietic system.

Preparation of Hematopoietic Cells Ex Vivo

Sources of hematopoietic stem and progenitor cells, which cells can be induced to proliferate according to one embodiment of the present invention, include but are not limited to bone marrow, fetal and neonatal blood (preferably from the umbilical cord and/or placenta), fetal liver, adult peripheral blood, neonatal thymus, and neonatal spleen. The foregoing list of sources is deemed to include cell samples (e.g., cryopreserved cells, cell lines, long-term cell cultures) derived therefrom. The source is mammalian, e.g., mouse, cow, horse, primate, monkey, and is most preferably human.

Techniques for obtaining such stem and progenitor cells are well known in the art.

For instance, in a particular embodiment, human bone marrow cells can be obtained from the posterior iliac crest by needle aspiration (see, e.g., Kodo et al., 1984, J. Clin. Invest. 73:1377-1384). Neonatal blood can be obtained at birth by direct drainage from the umbilical cord and/or by needle aspiration from the delivered placenta at the root and at distended veins (see U.S. Pat. Nos. 5,004,681 and 5,192,553). Fetal blood can be obtained, e.g., by taking it from the fetal circulation at the placental root with the use of a needle guided by ultrasound.

A method of the invention that comprises contacting hematopoietic stem and/or progenitor cells (or other hematopoietic cells) with a Therapeutic of the invention, optionally in the presence of another cytokine, can be carried out on unseparated, partially separated, or purified cell populations, before and/or after cryopreservation (and thawing) or in vitro culturing of such cell populations, before and/or after introduction of a recombinant gene, and any other desired manipulations of the cells. In a preferred aspect, samples (e.g., bone marrow or adult blood or neonatal blood) are subjected to physical and/or immunological cell separation procedures to enrich for hematopoietic stem and progenitor cells (e.g., prior to culturing in the presence of a Therapeutic of the invention to induce proliferation of the cells).

Various procedures are known in the art and can be used to enrich for stem and progenitor cells. These include, without limitation, equilibrium density centrifugation, velocity sedimentation at unit gravity, immune resetting and immune adherence, counterflow centrifugal elutriation, T lymphocyte depletion, and fluorescence-activated cell sorting, alone or in combination. Procedures have been reported for the isolation of very highly enriched populations of stem/progenitor cells. For example, U.S. Pat. No. 5,061,620 discloses a method for isolation of human hematopoietic stem cells. Murine CFU-S have been purified by several groups using slightly different procedures (Visser, et al., 1984, J. Exp. Med. 59:1576; Bauman et al., 1986, J. Cell. Physiol. 128:133; Lord and Spooncer, 1986, Lymphokine Res. 5:59). Studies using human (Emerson, et al., 1985, J. Clin. Invest. 76:1286) or murine (Nicola, et al., 1981, Blood 58:376) fetal liver cells have yielded highly enriched progenitor cells with up to 90% of them being colony forming cells for multi-, erythroid-, and granulocyte-macrophage lineages. CFU-E have also been very highly enriched (Nijhof, et al., 1983, J. Cell Biol. 96:386). Purification of adult mouse marrow CFU-GM with cloning efficiencies of up to 99% in semisolid medium has been accomplished by pretreatment of mice three days prior to sacrifice with cyclophosphamide, density separation of cells on Ficoll-Hypaque, and counterflow centrifugal clutriation (Williams et al., 1987, Exp. Hematol. 15:243). The resulting fraction of cells contained no detectable CFU-GEMM, BFU-E or CFU-MK, but up to 10% of the cells formed CFU-S measured at day 12. These procedures, or modifications thereof, can be used.

Human stem and progenitor cells are present in the non-adherent, low density, T lymphocyte-depleted fraction of bone marrow, spleen, and adult and cord blood cells. Low density (density less than 1.077 gm/cm³) cells can be separated by use of Ficoll-Hypaque or Percol (Broxmeyer, H. E., 1982, J. Clin. Invest. 69:632). In this procedure, the mature cells of the granulocytic series, which are not needed for transplantation, are removed in the dense fraction which goes to the bottom of the tube. An adherence/nonadherence separation protocol can also be used for enrichment of hematopoietic stem and progenitor cells.

It is also possible to use cell separation procedures that entail immunological recognition of cells. Stem and progenitor cells can be isolated by positive or negative selection using antibodies that recognize antigenic determinants on the surface of cells. One means is to separate the cells by using monoclonal antibodies which recognize cell surface determinants on these cells, in conjunction with separation procedures such as fluorescence-activated cell sorting or panning (Broxmeyer, et al., 1984 J. Clin. Invest. 73:939). Human hematopoietic stem and progenitor cells contain antigenic determinants that are not present on all other cells, which can be used in antibody selection protocols for enrichment purposes.

Within the human system, several antigens have been found on stem/progenitor cells. The first antigenic system studied intensively was that of the MHC class 11 antigens, especially HLA-DR. This antigen has been found on CFU-GEMM, BFU-E, and CFU-GM (Lu et al., 1983, Blood 61:250). Several investigators have suggested that HLA-DR are not found, or are present at a low density on cells earlier than CFU-GEMM (Moore et al., 1980, Blood 55:682; Keating et al., 1984, Blood 64:1159).

Groups of antibodies have been used to distinguish different progenitors of the granulocyte-macrophage lineage (Ferrero et al., 1983, Proc. Natl. Acad. Sci. 80:4114). Type 1 CFU-GM contribute all of the peripheral blood CFU-GM, as well as a small number of bone marrow CFU-GM. They express surface antigens recognized by S3-13 and S17-25 antibodies, but not by RIB19 and WGHS29-1 antibodies. Type 2 CFU-GM are present only in the marrow and react with S3-13, R11319, and WGIIS-29-1. Culture of type 1 CFU-GM in liquid culture generates type 2 CFU-GM. These antibodies have also been used to characterize CFU-GM from subjects with chronic myeloproliferative disorders (Ferrero et al., 1986, Cancer Res. 46:975).

Other antigens on human stem/progenitor cells include those reactive with the My10 (Strauss et al., 1986, Exp. Hematol. 14:879), 3C5 (Katz et al., 1985, Leukemia Res. 9:191), RFB-1 (Bodger et al., 1983, Blood 61:1006), 12-8 (Andrews et al., 1986, Blood 67:842), and L4F3 (Andrews et al., 1986, Blood 68:1030) antibodies. The antigen recognized by L4F3 is on CFU-GM, CFU-MK, BFU-E, and CFU-GEMM, but is apparently absent from cells which generate these progenitors in suspension culture (id.). The antigen recognized by the My10 antibody is CD34 (Civin, et al., U.S. Pat. No. 4,714,680). Two subsets of pluripotent hematopoietic stem cells have been reported, a CD34⁺ HLA-DR⁺ CD38⁻ subset and a more primitive CD34⁺ HLA-DRW CD38⁻ subset, with a gradual increase in CD38 expression as the hematopoietic cells proceed toward a more differentiated state (Huang and Terstappen, et al., 1992 Nature 360:745). The antigen recognized by another antibody, Myl 1 is expressed on CFU-GM, but not on BFU-E or CFU-GEMM (Strauss et al., 1986, Exp. Hematol. 14:935). Receptors for various lectins are also expressed on stem/progenitor cells.

To expand the numbers of the hematopoietic stem and/or progenitor cells, the hematopoietic stem and/or progenitor cells (or precursor cells thereof) are exposed, in a sterile environment, to or contacted with a composition comprising a Therapeutic of the invention for a sufficient time period, e.g., until the desired number of cells is obtained and the time period should be for as long as it is desired to keep cells self-renewing. Preferably, the cells are contacted with the Therapeutic, for example but not limited to, between about 0.1 and 1000 μg/kg while under appropriate culture conditions, for a time period in the range of 1-21 or, more preferably, 7-21 days.

A composition comprising a Therapeutic of the invention, to which the stem and/or progenitor cells are exposed according to the invention, may optionally also contain other growth factors and/or cytokines or cell culture materials, including but not limited to erythropoietin (Epo), Steel factor (SLF or SCF), IL-1, IL-3, IL-4, IL-6, IL-11, G-CSF, GM-CSF, FBS, adult PB plasma, alone or in combination. Preferably, factors are present that cause proliferation or, less preferably, differentiation of cells that are CFU-GEMM or earlier cells, e.g., IL-3, GM-CSF.

Contacting of the stem and progenitor cells with a Therapeutic may occur during cell culture and if so, the Therapeutic is preferably added to the cell culture medium being used to culture the hematopoietic stem and/or progenitor cells. Such culturing can be by any method known in the art, including, but not limited to, cells grown in culture dishes, test tubes, roller bottles, bioreactors, etc. Various protocols are known in the art for the growth in vitro of cord blood or bone marrow cells, and it is envisioned that such procedures, or modifications thereof, may be employed. The cell culture medium is supplemented to contain an effective concentration of the Therapeutic, for example, without limitation concentrations such as between about 0.1 and 1000 μg/kg.

Progeny cells of hematopoetic stem and progenitor cells can be generated in vivo; the differentiated progeny cells thus generated can be therapeutically useful. For example, in one embodiment, hematopoietic stem cells and/or CFU-GEMM progenitor cells, can be induced to differentiate into platelets. Such platelets can be used, for example, for infusion into a subject with thrombocytopenia, such as, but not limited to, the ITP associated with HIV infection. In another embodiment, granulocytes can be generated in vitro prior to infusion into a subject. One or more of the hematopoietic progeny cells can be generated in vitro, allowing for the in vitro production of blood components. In one embodiment, the generation of differentiated blood components is accompanied by expansion of the hematopoietic stem and progenitor cell pool in order to allow for production of a greater quantity of differentiated cells. Various growth factors can be used to promote expansion and/or differentiation of hematopoietic 15 stem and progenitor cells, such as cytokines (growth factors) including, but not limited to, G-CSF, CSF-1, IL-3, IL-5, tumor necrosis factor-R, and α-interferon. The blood components which are thus produced have uses which are not limited to therapeutic uses in vivo. For example, such progeny cells can be used in vitro, e.g., for the production and isolation of hematopoietic cell products such as growth factors, antibodies, etc.

A specific embodiment of the invention relates to a method of increasing the amount of hematopoietic cells, which method comprises contacting ex vivo a nonterminally differentiated hematopoietic cell with a composition comprising an amount of a Therapeutic of the invention effective to increase proliferation of the cell, under conditions suitable and for a time period sufficient to increase the numbers of said hematopoietic cell.

Assays for Induction of Hematopoietic Cell Proliferation

Any in vitro or in vivo assay known in the art to measure a pro-hematopoietic effect, e.g. the ability to induce hematopoietic cell proliferation in vitro or production of one or more hematopoietic cell types in vivo, can be used to test the efficacy of a Therapeutic of the invention (see e.g., examples herein).

A specific embodiment provides a method for screening a preparation comprising a Therapeutic of the invention, for pro-hematopoietic activity comprising assaying said preparation for the ability to induce an increase in hematopoietic cell numbers. In one embodiment, the preparation is screened by a method comprising measuring the number of colonies formed from hematopoietic stem or progenitor cells, which cells have been contacted with the preparation; and comparing the number of colonies formed from the cells contacted with the preparation with the number of colonies formed from cells not so contacted with the preparation, wherein a higher number of colonies formed from said contacted cells indicates that the preparation has pro-hematopoietic activity.

One can examine the effect of the Therapeutic on proliferation of hematopoietic cells in vitro. For example, to assay colony forming units, progenitor hematopoietic cells, e.g., such as CFU-GEMM, are cultured for an appropriate amount of time, such as about 5 to 20 days and preferably about 10 days, in the presence of (or otherwise exposed to) the Therapeutic to be tested, and then colony assays are performed to determine the number of colonies formed in comparison to the number of colonies formed by cells cultured in the absence of the Therapeutic. For example, hematopoietic progenitor cells can be isolated from bone marrow or cord blood, seeded in methylcellulose in the presence or absence of the Therapeutic (at various concentrations), and then colony numbers determined after about 10 days of culture. An increase in colony numbers in cells contacted with the Therapeutic indicates that the Therapeutic possesses the ability to induce proliferation of hematopoietic cells. Thus, for example, depending on the progenitor cell desired to be assayed, CFU-GM, CFU-GEMM, etc., assays can be done. Therapeutics can also be tested in vivo for activity in increasing the numbers of hematopoietic cells (see examples herein). Any animal model of an anemia can be similarly used for testing.

Therapeutics can be tested in human patients, preferably after tests in vitro and/or in vivo in an animal model, with hematopoietic deficiencies, for example but not limited to, deficiencies associated with HIV infection such as anemia, neutropenia, thrombocytopenia, or CD4⁺ T cell lymphocyte deficiency, for activity in increasing numbers of hematopoietic cells for which the patient is deficient. Briefly, the Therapeutic is administered, for example by intramuscular injection two to three times per week, to the subject with a hematopoietic deficiency. The subject's blood or bone marrow is assayed before and after treatment with the Therapeutic to monitor hematopoietic cell numbers. Therapeutics which cause an increase in hematopoietic cell numbers are useful for treatment of diseases and disorders associated with hematopoietic deficiencies. Therapeutics which cause a decrease in hematopoietic cell numbers are useful for treatment of diseases and disorders associated with hematopoietic surpluses.

Assays for hematopoietic cell proliferation in the blood or bone marrow can be accomplished by any method well known in the art. For example, blood can be drawn and blood cell numbers can be determined by routine clinical laboratory tests for red blood cells, platelets, neutrophils, lymphocytes, etc. Additionally, colony assays on isolated bone marrow can be performed to assess increases in stem or progenitor cells. For example, bone marrow can be sampled and bone marrow cells evaluated for stem and progenitor cell colony formation. Briefly, cells are seeded in methylcellulose, cultured for about 12 to 14 days, and then scored for colony formation where aggregates containing more than 50 cells are counted as a colony (see, e.g., Lunardi-Iskandar, Y et al., 1995, Nature 375:64). Bone marrow progenitors which can be evaluated by this colony assay include, but are not limited to, CFU-M, BFU-E and CFU-GM. As an alternative to colony assays for detection and quantitation of stem and/or progenitor cells, immunological detection methods can be employed, based on the antigens expressed by the particular cell type.

It is further contemplated that the present invention provides methods of treating or preventing hematopoietic disorders in mammals, preferably humans, comprising the administration of a therapeutically effective amount of a pharmaceutical composition comprising at least one resistin agonist, resistin polynucleotide, resistin functional fragment, resistin variant, and/or resistin polypeptide. Such methods are particularly useful for enhancing or stimulating hematopoiesis, erythropoiesis, leukopoiesis, thrombocytopoiesis, production of neutrophils, monocytes, granulocytes, and/or platelets by stimulating the proliferation and/or differentiation of progenitors of such cells, as needed in various conditions and/or situations. Such conditions and situations include, but are not limited to, the following:
(a) inadequate platelet production, such as aplastic anemia, refractory anemias, leukemia, preleukemia/myelodysplastic syndromes, megaloblastic anemia, chemotherapy or radiation therapy, and existing platelet deficiency or an expected platelet deficiency (e.g., because of planned surgery including, but not limited to, organ/bone marrow transplantations);
(b) abnormal platelet function, such as Glanzmann's thrombasthenia, acute/chronic leukemia, myeloproliferative disorders, uremia, platelet storage pool disease, Von Willebrand disease, and postoperative cardiovascular dysfunction, and
(c) other blood coagulation disorders such as afibrinogenemia or wounds of any origin.

The generic term for platelet deficiency is thrombocytopenia, and hence the methods and compositions of the present invention are generally available for treating thrombocytopenias. Thrombocytopenias (platelet deficiencies) may be present for various reasons, including chemotherapy, radiation therapy, surgery, accidental blood loss, and other specific disease conditions. Exemplary specific disease conditions that involve thrombocytopenia and may be treated in accordance with this invention are: aplastic anemia, idiopathic thrombocytopenia, and certain metastatic tumors which result in thrombocytopenia. Also, certain treatments for Acquired Immunodeficiency Syndrome (AIDS) result in thrombocytopenia (e.g., AZT). Certain wound healing disorders might also benefit from an increase in platelet numbers.

With regard to anticipated platelet deficiencies, e.g., due to future surgery, the resistin polypeptide, resistin agonist or variants thereof could be administered several days to several hours prior to the need for platelets. With regard to acute situations, e.g., accidental and massive blood loss, the resistin polypeptide, agonist, and/or variants thereof could be administered along with blood or purified platelets. A preferred embodiment of the present invention provides methods of treating or preventing hematopoietic disorders including, but not limited to, anemia and disorders commonly associated with anemia comprising the administration to a mammal in need of such treatment a therapeutically effective amount of a pharmaceutical composition comprising an isolated resistin polypeptide having the sequence as shown in SEQ ID NO: 2-7, or a variant thereof.

The present invention further provides a pharmaceutical formulation that comprises at least one resistin agonist, resistin polynucleotide, resistin polypeptide, resistin functional fragment, resistin variant, and/or resistin composition together with one or more pharmaceutically acceptable diluents, carriers, or excipients therefor.

The present invention also provides a method of treating or preventing hematopoietic disorders including, but not limited to, anemia and/or disorders commonly associated with anemia, comprising the administration to a mammal in need thereof of a therapeutically effective amount of a resistin composition wherein said composition has at least one activity, such as, but not limited to, inducing differentiation and/or proliferation of CFU-GEMM, erythroid, CFU-GM and/or megakaryocyte progenitor cells. A resistin polypeptide can thus be screened for a corresponding activity according to these effects.

The invention further provides for the use of a resistin agonist, resistin nucleic acid, resistin polypeptide, resistin functional fragment and/or resistin variant in the manufacture of a medicament for the treatment or prevention of anemia, and disorders associated with such conditions.

The novel methods contemplated by the present invention include methods of using resistin polynucleotides and resistin polypeptides as shown in SEQ ID NOS: 1 and 2-7, respectively, and functional fragments thereof, as well as resistin polynucleotide variants and/or resistin polypeptide variants that further comprise one or more substitutions, deletions, insertions, inversions, additions, or changes in glycosylation sites or patterns yet have substantially similar biological activities and/or pharmaceutically desired properties as the corresponding unmodified resistin polynucleotide or resistin polypeptide to treat mammals, preferably humans, to alter the hematopoietic cell composition of those in need of such treatment.

Resistin Polypeptide Variants

Resistin polypeptide variants comprise about 95% or greater sequence identity with the sequence shown in SEQ ID NO: 2-7. In one embodiment of the present invention, a single amino acid change is made within the resistin polypeptide which has an amino acid sequence as shown in SEQ ID NO: 2-7. Alternatively, at least two changes are made within the resistin polypeptide sequences; alternatively, at least three changes are made within the resistin polypeptide sequences; alternatively, at least four changes up to at least 10 changes are made within a resistin polypeptide sequence (SEQ ID NOS: 2-7). An amino acid change may be an insertion, substitution and/or deletion. The changes may or may not be contiguous and may be at the N- or C-terminus or internal. As the skilled artisan understands, many substitutions, and/or other changes to a protein's sequence or structure, can be made without substantially affecting the biological activity or characteristics of the polypeptide. For example, making conservative amino acid substitutions, or changing one amino acid for another from the same class of amino acids, (e.g., negatively charged residues, positively charged residues, polar uncharged residues, and non-polar residues, or any other classification acceptable in the art) are often made without significant effects upon function. Modifications of the resistin polypeptide sequence as shown in SEQ ID NOs: 2-7 made in accordance with Table I are expected to result in resistin variants that retain the same or substantially similar biological activity as the unmodified resistin polypeptide based on art-recognized substitutability of certain amino acids (see, e.g., M. Dayhoff, In Atlas of Protein Sequence and Structure, Vol. 5, Supp. 3, pgs 345-352, 1978) and are also contemplated as being useful in the methods of the present invention.

One factor that can be considered in making such amino acid changes is the hydropathic index of amino acids. The importance of the hydropathic amino acid index in conferring interactive biological function on a protein has been discussed by Kyte and Doolittle (1982, J. Mol. Biol., 157:105). It is accepted that the relative hydropathic character of amino acids contributes to the secondary structure of the resultant protein. This, in turn, affects the interaction of the protein with molecules such as enzymes, substrates, receptors, ligands, DNA, antibodies, antigens, etc. As is known in the art, certain amino acids in a peptide, polypeptide, or protein can be substituted for other amino acids having a similar hydropathic index or score and produce a resultant peptide, etc., having similar biological activity, i.e., which still retains biological functionality. In making such changes, it is preferable that amino acids having hydropathic indices within ±2 are substituted for one another. More preferred substitutions are those wherein the amino acids have hydropathic indices within ±1. Most preferred substitutions are those wherein the amino acids have hydropathic indices within ±0.5.

Like amino acids can also be substituted on the basis of hydrophilicity. U.S. Pat. No. 4,554,101 discloses that the greatest local average hydrophilicity of a protein, as governed by the hydrophilicity of its adjacent amino acids, correlates with a biological property of the protein. The following hydrophilicity values have been assigned to amino acids: arginine/lysine (+3.0); aspartate/glutamate (+3.0±1); serine (+0.3); asparagine/glutamine (+0.2); glycine (0); threonine (−0.4); proline (−0.5±1); alanine/histidine (−0.5); cysteine (−1.0); methionine (−1.3); valine (−1.5); leucine/isoleucine (−1.8); tyrosine (−2.3); phenylalanine (−2.5); and tryptophan (−3.4). Thus, one amino acid in a peptide, polypeptide, or protein may be substituted by another amino acid having a similar hydrophilicity score and still produce a resultant peptide, etc., having similar biological activity, i.e., still retaining correct biological function. In making such changes, amino acids having hydropathic indices within ±2 are preferably substituted for one another, those within ±1 are more preferred, and those within ±0.5 are most preferred.

As outlined above, amino acid substitutions in a resistin polypeptide can be based on the relative similarity of the amino acid side-chain substituents, for example, their hydrophobicity, hydrophilicity, charge, size, etc. Exemplary substitutions that take various of the foregoing characteristics into consideration in order to produce conservative amino acid changes resulting in silent changes within the present peptides, etc., can be selected from other members of the class to which the naturally occurring amino acid belongs. Amino acids can be divided into the following four groups: (1) acidic amino acids; (2) basic amino acids; (3) neutral polar amino acids; and (4) neutral non-polar amino acids. Representative amino acids within these various groups include, but are not limited to: (1) acidic (negatively charged) amino acids such as aspartic acid and glutamic acid; (2) basic (positively charged) amino acids such as arginine, histidine, and lysine; (3) neutral polar amino acids such as glycine, serine, threonine, cysteine, cystine, tyrosine, asparagine, and glutamine; and (4) neutral non-polar amino acids such as alanine, leucine, isoleucine, valine, proline, phenylalanine, tryptophan, and methionine.

Resistin variants having a biological activity, in vivo or in vitro (i.e., ex vivo), that is similar or identical to one described herein, for example, the ability to increase the number of CFU-GEMM cells or to induce or enhance differentiation and/or proliferation of erythroid and/or megakaryocyte progenitor cells, are also useful in the methods of the present invention and as such are contemplated by the present invention. Resistin variants, while being functionally related, by definition include amino acid sequences that differ in one or more positions up to about 5 positions from the sequence as shown in SEQ ID NOs: 2-7. Resistin variants that are useful in the methods of the present invention can be generated by deletion, insertion, inversion, and/or substitution of one or more amino acid residues in said resistin polypeptide. Such resistin variants can generally be made by solid phase or recombinant techniques in which, for example, single or multiple conservative amino acid substitutions are made, according to Table 1.

Generally, in the case of multiple substitutions, it is preferred that at least 95%, more preferably at least 96%, 97%, 98% or 99% of the residues of a resistin variant are identical to the corresponding contiguous sequence as shown in SEQ ID NO: 2, 3, 4, 5, 6 or 7. Examples of most preferred resistin variants include the resistin variant as shown in SEQ ID NO: 3.

Another class of resistin variant that may be useful in the methods of the present invention includes resistin polypeptides as defined herein further comprising at least one oligopeptide or amino acid added onto the N-terminus and/or C-terminus. An “oligopeptide” is a chain of from two to about twenty-five amino acids connected at their N- and C-termini by peptide bonds. Suitable oligopeptides and amino acids are those that do not significantly decrease the biological activity of the resistin polypeptide as defined herein and do not substantially detract from the desired pharmaceutical and pharmacological properties of the resistin polypeptide. A preferred example of such a modification includes a resistin polypeptide as defined herein further comprising a leader sequences found in other polypeptides, such as pretrypsinogen leader sequence.

The resistin polypeptides as defined herein can also be expressed and used in a modified form, such as a fusion protein or a “tagged” protein. A resistin fusion protein represents a hybrid protein molecule not found in nature comprising a translational fusion or enzymatic fusion in which two or more different proteins, fragments, or variants thereof are covalently linked on a single polypeptide chain. The two or more proteins may optionally be separated by a linker sequence to allow for independent activity of each of the fused proteins. Human serum albumin, the C-terminal domain of thrombopoietin, the C-terminal extension peptide of hCG, and/or a Fc fragment are examples of proteins which could be fused with resistin polypeptides, resistin fragments and/or resistin variants for use in the present invention. As used herein, “Fc fragment” of an antibody has the meaning commonly given to the term in the field of immunology. Specifically, this term refers to an antibody fragment which binds complement and is obtained by removing the two antigen binding regions (the Fab Fragments) from the antibody. Thus, the Fc fragment is formed from approximately equal sized fragments from both heavy chains, which associate through non-covalent interactions and disulfide bonds. The Fc Fragment includes the hinge regions and extends through the CH2 and CH3 domains to the C-terminus of the antibody. Procedures for preparing fusion proteins are disclosed in EP 394827, Tranecker et al., Nature 331:84, 1988, and Fares, et al., Proc. Natl. Acad. Sci. 89:4304,1992.

Many fusion proteins can be secreted by virtue of heterologous secretion signals in regions that can be removed prior to final preparation of the polypeptide. Such methodologies are well known in the art. In a preferred process for protein expression and subsequent purification, the resistin gene can be modified at the 5′ end to incorporate several histidine residues at the amino terminus of the resistin protein resulting from its expression. This “histidine tag” enables a single-step protein purification method referred to as “immobilized metal ion affinity chromatography” (IMAC), essentially as described in U.S. Pat. No. 4,569,794, which is incorporated herein by reference. The IMAC method enables rapid isolation of substantially pure recombinant resistin protein starting from a crude extract of cells that express a modified recombinant protein, as described above.

A resistin polypeptides and/or resistin variants useful in the present invention as well as the nucleic acids encoding them may also be defined with reference to a percent identity similarity to either SEQ ID NO: 2, 3 or SEQ ID NO: 1. Sequence identity refers to a comparison between two molecules using standard algorithms well known in the art. Although any suitable sequence comparison algorithm can be used for this purpose, for illustration, this embodiment shall be described with reference to the well-known Smith-Waterman algorithm using SEQ ID NO: 2 as the reference sequence to define percent identity to a comparator sequence. When sequence identity is used with reference to a polypeptide, either the entire polypeptide may be used in the comparison or instead only a defined sub-region thereof.

The choice of parameter values for matches, mismatches, and inserts or deletions is arbitrary. A preferred set of values for use with the Smith-Waterman algorithm is set forth in the “maximum similarity segments” approach, which uses values of 1 for a matched residue, and −⅓ for a mismatched residue (see, Waterman, Bulletin of Matheniatical Biology, 46, 473-500, 1984). Insertions and deletions (indels), x, are weighted as follows:

Xₖ=1+k/3 where k is the number of residues in a given insert or deletion. For example, a comparator sequence that has 20 substitutions and 3 insertions relative to the 250 residue reference protein sequence would have an identity of: [(1×250)−(⅓×20)−(1+3/3)]/250=96% identical.

Since resistin variants can be produced easily by conventional recombinant or solid phase synthetic techniques known in the art, the methods of the present invention contemplates the use of resistin polynucleotide and resistin polypeptide variants in the methods of the present invention to the extent that such variants have at least 70%, 80%, 90%, 95%, 96%, 97%, 98% or 99% identity to a contiguous sequence of nucleotides or amino acids as shown in SEQ ID NO: 1 or 2, respectively, while retaining substantially similar activity as the corresponding resistin polynucleotide or resistin polypeptide.

Resistin Polypeptide Synthesis

Functional fragments of resistin polypeptides and resistin variants may be generated by any number of suitable techniques, including chemical synthesis of any portion of SEQ ID NO: 2, proteolytic digestion of resistin polypeptides or resistin variants, or most preferably, by recombinant DNA mutagenesis techniques well known to the skilled artisan. Functional fragments of the proteins disclosed herein may be produced as described above, preferably using cloning techniques to engineer smaller versions of the intact gene, deleting sequence from the 5′ end, the 3′ end, from both ends, or from an internal site. Fragments may be tested for biological activity using any suitable assay, for example, the ability to induce and/or enhance differentiation and/or proliferation of erythroid, megakaryocyte or CFU-GM or CFU-GEMM progenitor cells in vivo or in vitro.

Those skilled in the art will recognize that the resistin gene could be obtained by a plurality of recombinant DNA techniques including, for example, as described in international application WO 99/27103, incorporated herein, or by hybridization, polymerase chain reaction (PCR) amplification, or de novo DNA synthesis.(See e.g., Sambrook, et al. Molecular Cloning: A Laboratory Manual 2d Ed. Chap. 14 (1989)). Methods for constructing cDNA libraries in a suitable vector such as a plasmid or phage for propagation in prokaryotic or eukaryotic cells are well known to those skilled in the art. Suitable cloning vectors are well known and are widely available.

Skilled artisans will recognize that the proteins used in the present invention can be synthesized by a number of different methods, such as chemical methods well known in the art, including solid phase peptide synthesis or recombinant methods. Both methods are described in U.S. Pat. No. 4,617,149, incorporated herein by reference. The proteins useful in the present invention can also be produced by recombinant DNA methods using the cloned resistin gene. Recombinant methods are preferred if a high yield is desired. Expression of the cloned gene can be carried out in a variety of suitable host cells, well known to those skilled in the art. For this purpose, the resistin gene is introduced into a host cell (e.g., prokaryote, mammalian, yeast, SF9) by any suitable means, well known to those skilled in the art. While chromosomal integration of the cloned gene is within the scope of the present invention, it is preferred that the gene be cloned into a suitable extra-chromosomally maintained expression vector so that the coding region of the resistin gene is operably-linked to a constitutive or inducible promoter suitable for the expression system of choice. The recombinantly-produced protein may be purified from cellular extracts of transformed cells by means known well in the art.

The resistin polypeptides used in the methods of the present invention may be synthesized either by direct expression or as a fusion protein comprising the resistin polypeptide of interest as a translational fusion with another protein or peptide which optionally may be removable by enzymatic or chemical cleavage. It is often observed in the production of certain peptides in recombinant systems that expression as a fusion protein prolongs the life span, increases the yield of the desired peptide, provides a convenient means of purifying the protein. This is particularly relevant when expressing mammalian proteins in prokaryotic hosts. A variety of peptidases which cleave a polypeptide at specific sites or digest the peptides from the amino- or carboxy-termini of the peptide chain are known. Furthermore, particular chemicals (e.g. cyanogen bromide) will cleave a polypeptide chain at specific sites. The skilled artisan will appreciate the modifications necessary to the amino acid sequence (and synthetic or semi-synthetic coding sequence if recombinant means are employed) to incorporate site-specific internal cleavage sites (See e.g., P. Carter, “Site Specific Proteolysis of Fusion Proteins”, Chapter 13, in Protein Purification: From Molecular Mechanisms to Large Scale Processes, American Chemical Society, Washington, D.C. (1990)).

The full-length resistin cDNA (as shown in SEQ ID NO: 1) and related nucleic acid molecules that encode e.g., SEQ ID NOs: 2-7 and/or fragments and/or variants thereof, may be produced by chemical synthetic methods. The synthesis of nucleic acids is well known in the art. Fragments of the DNA sequence corresponding to the resistin gene may be generated using a conventional DNA synthesizing apparatus using phosphoramidite chemistry, thereafter ligating the fragments so as to reconstitute the entire gene. Alternatively, phosphotriester chemistry may be employed to synthesize the nucleic acids of this invention.

In an alternative methodology, namely PCR, the DNA sequences disclosed and described herein, comprising, for example, a portion or all of SEQ ID NO: 1 can be produced from a plurality of starting materials. For example, starting with a cDNA preparation (e.g. cDNA library) derived from a tissue that expresses the resistin gene, suitable oligonucleotide primers complementary to SEQ ID NO: 1 or to any sub-region therein, or adjacent region. Using PCR, any region of the resistin gene can be targeted for amplification such that full or partial length gene sequences may be produced.

The present invention still further relates to methods for identifying compounds which modulate the expression of the mammalian resistin gene and/or the synthesis or activity of mammalian resistin gene products. Such compounds include therapeutic compounds that can be used as pharmaceutical compositions to reduce or eliminate the symptoms of mammalian hematopoietic disorders such as anemia. Cellular and non-cellular assays are described that can be used to identify compounds that interact with the resistin gene and/or gene product, e.g., modulate the activity of the resistin gene and/or bind to the resistin gene product. Such cell-based assays of the invention utilize cells, cell lines, or engineered cells or cell lines that express the resistin gene product.

First, cell-based systems can be used to identify compounds that may act to ameliorate hematopoietic disorder symptoms. Such cell systems can include, for example, recombinant or non-recombinant cell, such as cell lines, that express the resistin gene. In utilizing such cell systems, cells that express resistin may be exposed to a compound suspected of exhibiting an ability to ameliorate hematopoietic disorder symptoms, at a sufficient concentration and for a sufficient time to elicit such an amelioration of such symptoms in the exposed cells. After exposure the cells can be assayed to measure alterations in the expression of the resistin gene, e.g., by assaying cell lysates for resistin mRNA transcripts (e.g., by Northern analysis) or for resistin gene products expressed by the cell. Compounds that modulate expression of the resistin gene are good candidates as therapeutics.

In addition, animal-based systems or models for a mammalian hematopoietic disorder, for example, transgenic mice containing a human or altered form of resistin gene, may be used to identify capable of ameliorating symptoms of the disorder. Such animal models may be used as test substrates for the identification of drugs, pharmaceuticals, therapies and interventions. For example, animal models may be exposed to a compound suspected of exhibiting an ability to ameliorate symptoms, at a sufficient concentration and for a sufficient time to elicit such an amelioration of the symptoms of the hematopoietic disorder. An animal's response to a particular treatment may be monitored by assessing reductions in the symptoms attributable to the disorder. Treatments that favorably affect hematopoietic disorder-like symptoms may be considered as candidates for human therapeutic intervention in such a disorder. Dosages of test agents may be determined by deriving dose-response curves

In one embodiment methods of the present invention comprise contacting a compound to a cell, measuring the level of resistin gene expression, gene product expression, or gene product activity, and comparing the level to the level of resistin gene expression, gene product expression, or gene product activity produced by the cell in the absence of the compound. If the level obtained in the presence of the compound differs from that obtained in its absence, a compound that modulates the expression of the mammalian resistin gene and/or the synthesis or activity of mammalian resistin gene products has been identified.

Alternative embodiment methods of the present invention comprise administering a compound to a host, (e.g., a wild-type or transgenic animal that expresses a resistin transgene or a resistin variant transgene) and measuring the level of resistin gene expression, gene product expression, or gene product activity. The measured level is compared to the level of resistin gene expression, gene product expression, or gene product activity in a host that is not exposed to the compound. If the level obtained when the host is exposed to the compound differs from that obtained when the host is not exposed to the compound, a compound that modulates either the expression of the mammalian resistin gene, the synthesis or activity of the resistin gene product, the compound may be used in the methods of the present invention.

Methods of the present invention can comprise, for example administering compounds which modulate the expression of a mammalian resistin gene and/or the synthesis and/or the activity of a mammalian resistin gene product, so that symptoms of a hematopoietic disorder are ameliorated. Alternatively, in those instances whereby the mammalian hematopoietic disorder results from resistin gene mutations, such methods can comprise supplying the mammal with a nucleic acid molecule encoding an unimpaired resistin gene product such that an unimpaired resistin gene product is expressed and symptoms of the disorder are ameliorated.

Another embodiment of the present invention includes a method for the treatment of mammalian hematopoietic disorder comprising the administration of a cell comprising a nucleic acid molecule that encodes an unimpaired resistin gene product to mammal in need of such treatment such that the cell expresses a unimpaired resistin gene product and/or polypeptide encoded thereby, and symptoms of the disorder are ameliorated.

Pharmaceutical Compositions

For therapeutic utility, an effective amount of resistin agonist, resistin polynucleotide, resistin polypeptide, resistin functional fragment and/or resistin variant is administered to a mammal in need thereof in a dose between about 0.1 and 1000 μg/kg. In practicing the methods contemplated by this invention, the resistin agonists, resistin polynucleotides, resistin polypeptides, resistin functional fragments, resistin variants, and/or resistin compositions thereof as defined herein can be administered in multiple doses per day, in single daily doses, in weekly doses, or at any other regular interval. The amount per administration and frequency of administration will be determined by a physician and depend on such factors as the nature and severity of the disease, and the age and general health of the patient.

The present invention also provides a pharmaceutical resistin composition comprising as the active agent an resistin agonist, resistin polynucleotide, resistin polypeptide, resistin functional fragment, resistin variant, and/or a pharmaceutically acceptable non-toxic salt thereof, and a pharmaceutically acceptable solid or liquid carrier. For example, at least one resistin agonist, resistin polynucleotide, resistin polypeptide, resistin functional fragment, resistin variant can be admixed with conventional pharmaceutical carriers and excipients, and used in the form of tablets, capsules, elixirs, suspensions, syrups, wafers, parenteral formulations, and the like. The resistin compositions will contain from about 0.1% to 90% by weight of the active resistin agonist, resistin polynucleotide, resistin polypeptide, resistin functional fragment and/or resistin variant, and more generally from about 10% to 30%. The resistin compositions may contain common carriers and excipients such as corn-starch or gelatin, lactose, sucrose, microcrystalline cellulose, kaolin, mannitol, dicalcium phosphate, sodium chloride, and alginic acid.

As a general proposition, the total pharmaceutically effective amount of a resistin agonist, resistin polynucleotide, resistin polypeptide, resistin functional fragment and/or resistin variant administered parenterally to a patient per dose will be in the range of about 1 μg/kg/day to 10 mg/kg/day, particularly 2 mg/kg/day to 8 mg/kg/day, more particularly 2 mg/kg/day to 4 mg/kg/day, even more particularly 2.2 mg/kg/day to 3.3 mg/kg/day, and finally 2.5 mg/kg/day, although, as noted above, this will be subject to therapeutic discretion. More preferably, this dose is at least 0.01 mg/kg/day. If given continuously a resistin agonist, resistin polynucleotide, resistin polypeptide, resistin functional fragment and/or resistin variant is typically administered at a dose rate of about 1 μg/kg/hour to about 50 μg/kg/hour, either by 1-4 injections per day or by continuous subcutaneous infusions, for example, using a mini-pump. An intravenous bag solution may also be employed. The length of treatment needed to observe changes and the interval following treatment for responses to occur appears to vary depending on the desired effect.

Pharmaceutical compositions containing a resistin agonist, resistin polynucleotide, resistin polypeptide, resistin functional fragment and/or resistin variant may be administered orally, rectally, intracranially, parenterally, intracistemally, intravaginally, intraperitoneally, topically (as by powders, ointments, drops or transdermal patch), transdermally, intrathecally, bucally, or as an oral or nasal spray. By “pharmaceutically acceptable carrier” is meant a non-toxic solid, semisolid or liquid filler, diluent, encapsulating material or formulation auxiliary of any type. The term “parenteral” as used herein includes, but is not limited to, modes of administration which include intravenous, intramuscular, intraperitoneal, intrastemal, subcutaneous and intra-articular injection, infusion and implants comprising a resistin agonist, resistin polynucleotide, resistin polypeptide, resistin functional fragment and/or resistin variant.

The compounds can be formulated for oral or parenteral administration. A preferred parenteral formulation for subcutaneous administration would comprise a buffer (phosphate, citrate, acetate, borate, TRIS), salt (NaCl, KCl), divalent metal (Zn, Ca), and isotonicty agent (glycerol, mannitol), detergent (Polyoxyethylene sorbitan fatyy acid esters, poloxamer, ddicusate sodium, sodium lauryl sulfate), antioxidants (ascorbic acid), and antimicrobial agent (phenol, m-cresol, alcohol, benzyl alcohol, butylparben, methylparaben, ethylparaben, chlorocresol, phenoxyethanol, phenylethyl alcohol, propylparaben.

For intravenous (IV) use, a resistin agonist, resistin polynucleotide, resistin polypeptide, resistin functional fragment and/or resistin variant is administered in commonly used intravenous fluid(s) and administered by infusion. Such fluids, for example, physiological saline, Ringer's solution or 5% dextrose solution can be used.

For intramuscular preparations, a sterile formulation, preferably a suitable soluble salt form of a resistin agonist, resistin polynucleotide, resistin polypeptide, resistin functional fragment and/or resistin variant such as the hydrochloride salt, can be dissolved and administered in a pharmaceutical diluent such as pyrogen-free water, physiological saline, or a 5% glucose solution. A suitable insoluble form of the compound may be prepared and administered as a suspension in an aqueous base or a pharmaceutically acceptable oil base, e.g. an ester of a long chain fatty acid.

A resistin agonist, resistin polynucleotide, resistin polypeptide, resistin functional fragment, and/or resistin variant is also suitably administered by sustained-release systems. Suitable examples of sustained-release compositions include semi-permeable polymer matrices in the form of shaped articles, e.g., films, or microcapsules. Sustained-release matrices include polylactides (U.S. Pat. No. 3,773,919, EP 58,481), copolymers of L-glutamic acid and gamma-ethyl-L-glutamate, poly (2-hydroxyethyl methacrylate), ethylene vinyl acetate or poly-D-(−)-3-hydroxybutyric acid (EP 133,988). Other sustained-release compositions also include liposomally entrapped modified resistin polypeptides and/or fragments thereof and/or variants thereof. Ordinarily, the liposomes are of the small (about 200-800 Angstroms) unilamellar type in which the lipid content is greater than about 30 mol. percent cholesterol, the selected proportion being adjusted for the optimal therapy.

For parenteral administration, in one embodiment, the resistin agonist, resistin polynucleotide, resistin polypeptide, resistin functional fragment and/or resistin variant may be formulated generally by mixing it at the desired degree of purity, in a unit dosage injectable form (solution, suspension, or emulsion), with a pharmaceutically acceptable carrier (i.e., one that is non-toxic to recipients at the dosages and concentrations employed and is compatible with other ingredients of the formulation). Preferably, the formulation does not include oxidizing agents and other compounds that are known to be deleterious to polypeptides.

Generally, the formulations are prepared by contacting resistin agonist, resistin polynucleotide, resistin polypeptide, resistin functional fragment and/or resistin variant uniformly and intimately with liquid carriers or finely divided solid carriers or both. Then, if necessary, the product is shaped into the desired formulation. Preferably the carrier is a parenteral carrier, more preferably a solution that is isotonic with the blood of the recipient. Examples of such carrier vehicles include water, saline, Ringer's solution, and dextrose solution. Non-aqueous vehicles such as fixed oils and ethyl oleate are also useful herein, as well as liposomes.

The carrier suitably contains minor amounts of additives such as substances that enhance isotonicity and chemical stability. Such materials are non-toxic to recipients at the dosages and concentrations employed, and include buffers such as phosphate, citrate, succinate, acetic acid, and other organic acids or their salts; antioxidants such as ascorbic acid; low molecular weight (less than about ten residues) polypeptides, e.g., polyarginine or tripeptides; proteins, such as serum albumin, gelatin, or immunoglobulins; hydrophilic polymers such as polyvinylpyrrolidone; amino acids, such as glycine, glutamic acid, aspartic acid, or arginine; monosaccharides, disaccharides, and other carbohydrates including cellulose or its derivatives, glucose, manose, or dextrins; chelating agents such as EDTA; sugar alcohols such as mannitol or sorbitol; counterions such as sodium; and/or nonionic surfactants such as polysorbates, poloxamers, or PEG.

A resistin agonist, resistin polynucleotide, resistin polypeptide, resistin functional fragment and/or resistin variant is typically formulated in such vehicles at a concentration of about 0.1 mg/ml to 100 mg/ml, preferably 1-10 mg/ml, at apH of about 3 to 8. It will be understood that the use of certain of the foregoing excipients, carriers, or stabilizers will result in the formation of salts of the particular active ingredient(s).

Compositions to be used for therapeutic administration must be sterile. Sterility is readily accomplished by filtration through sterile filtration membranes (e.g., 0.2 micron membranes). Therapeutic compositions generally are placed into a container having a sterile access port, for example, an intravenous solution bag or vial having a stopper pierceable by a hypodermic injection needle.

Pharmaceutically useful resistin-comprising compositions ordinarily will be stored in unit or multi-dose containers, for example, sealed ampoules or vials, as an aqueous solution or as a lyophilized formulation for reconstitution. As an example of a lyophilized formulation, 10-ml vials are filled with 5 ml of sterile-filtered 1% (w/v) aqueous solution of one of a resistin composition, and the resulting mixture is lyophilized. The infusion solution is prepared by reconstituting the lyophilized polypeptide using bacteriostatic water for injection.

The invention also provides a pharmaceutical pack or kit comprising one or more containers filled with one or more of the ingredients of the pharmaceutical compositions of the invention. Associated with such container(s) can be a notice in the form prescribed by a governmental agency regulating the manufacture, use or sale of pharmaceuticals or biological products, which notice reflects approval by the agency of manufacture, use or sale for human administration. Progress of the treated patient can be monitored by assays provided herein or otherwise known in the art.

The following examples more fully describe the present invention. Those skilled in the art will recognize that the particular reagents, equipment, and procedures described are merely illustrative and are not intended to limit the present invention in any manner.

### EXAMPLES

### Example 1

### Expression of Human Resistin

Human resistin cDNA clone was obtained from Incyte (Incyte clone 1668415). The open reading frame for human resistin gene was amplified by PCR and cloned into the pPR1 expression vector (Lilly) with or without Flag-His tag using the following primers:

Forward:

(SEQ ID NO: 8)

5′-GATCGGCGCGCCAGCCACCATGAAAGCTCTCTGTCTCCT-3′
Reverse:

(SEQ ID NO: 9)

5′-CGCGATATCGGGCTGCACACGACAGCAGC-3.

To express human resistin, 293E cells were transfected with the recombinant pPR1 plasmid containing the resistin gene using standard procedures. The resistin protein expressed from the transfected cells were identified by Western analysis using anti-Flag antibody (Sigma). The Western result showed that the resistin protein was a secreted molecule with molecular weight of about 10-14 kD. The non-tagged resistin could be detected by polyclonal antibody against tagged resistin (raised in rabbit).

Cell culture media containing resistin (secreted from cells expressing FLIS-tagged-resistin) is concentrated in an Amicon ProFlux M12 tangential filtration system using an Amicon S3Y10 UF membrane. A Ni-NTA column (20 ml) was prepared and equilibrated with 200 ml of PBS (pH 7.5) at a flow rate of 8 ml/min. The 293 media expressing resistin (20-30 L) was loaded onto the Ni-NTA column. The column is washed with 200 ml of PBS. The proteins are eluted with a 500 ml gradient of 0 to 0.25 M imidazol in PBS (pH7.5). 8 ml fractions are collected, and the fractions analyzed on SDS-PAGE. The fractions containing resistin are identified and pooled. 5 mM EDTA is added to the pooled sample to chelate Ni. The sample is dialyzed at first against 4 L PBS with 5 changes of the dialysis buffer, then against 25 mM Hepes (pH 7.0) containing 50 mM NaCl. The dialyzed sample is loaded onto a SP column equilibrated with 25 mM Hepes containing 50 mM NaCl (pH 7.0). The flow through fraction is collected which contains resistin. The sample is dialyzed against 4 L PBS, and concentrated to 0.5 mg/ml. Purified resistin is analyzed on SDS-PAGE (4-20%) in the absence or presence of 20 mM DTT. The SDS-gel is stained with Coomassie Blue. Resistin appears as a 28 kDa band (dimer) in the absence of DTT, and a 14 kDa band in the presence of DTT (monomer). The protein was also analyzed by electrospray mass spec. The protein primarily appeared to form dimmers or dimmer adduct multimers.

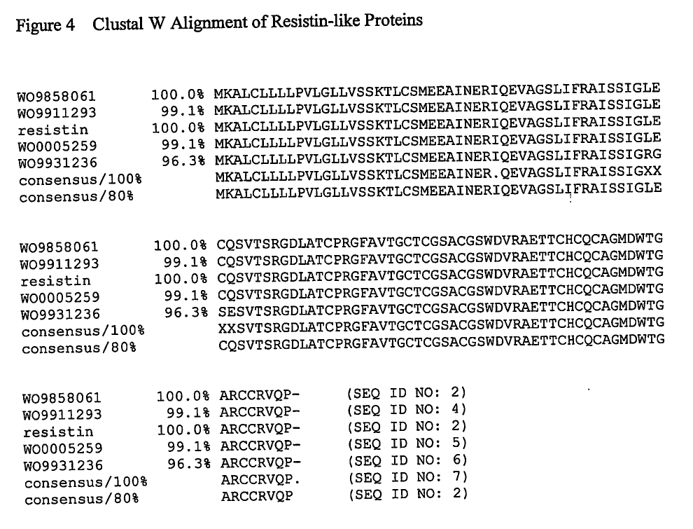

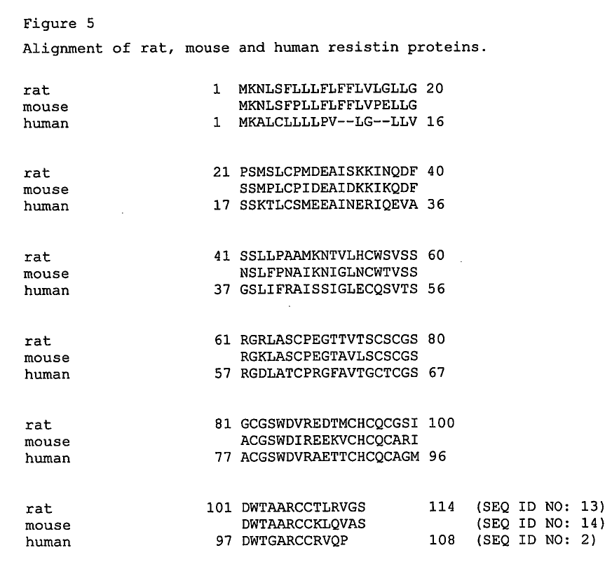

Human resistin has 108 amino acids, 11 cysteine residues and has a theoretical molecular weight of 11,836 Da and a Maldi spec molecular weight of 11,834 daltons (monomer) and 23,648 daltons (dimmer). It has no potential N-linked glycosylation sites. An alignment of the various reported amino acid sequences of human resistin is shown in FIG. 4 herein and an alignment of the mouse, rat and human amino acid sequences of resistin is shown in FIG. 5.

### Example 2

### Northern Blot Detection of Resistin in Human Tissues

DIG-labeled probe was synthesized using PCR DIG probe synthesis kit (Roche) following the manufacturer's instruction. Briefly, DIG-labeled probe was PCR-amplified from the resistin template (resistin-Glag-His/pPR1) using the same primers for cloning the resistin open reading frame described in Example 1 above. RNA blots were purchased from Clontech or produced according to standard methods loading 1-5 μg mRNA per lane. The blot was hybridized with DIG-labeled resistin probe and signal was detected by CDP-Star reagent (Roche) following the manufacturer's instruction. Twenty-eight tissues were examined for human resistin expression. An approximately 0.8 kb RNA was readily detected in fetal liver and adult bone marrow, and weaker expression was observed in lung and peripheral blood leukocytes. Human resistin could not be detected in white fat. The result suggests that human resistin may play a role in hematopoietic system as a stem cell factor or cytokine

### Example 3

### In Situ Hybridization

Probe was prepared for in situ hybridization by cloning the human resistin gene into the plasmid Bluescript KS+II. An RNA probe was synthesized by in vitro transcription using the DIG RNA labeling kit (Roche) according to manufacturer's protocol. The 10× NTP labeling mix was replaced however with fluorescein RNA labeling mix (Roche). For detection of human resistin expression in messenchymal cells, a messenchymal cell monolayer was fixed on a chamber slide. After hydration, the slide was hybridized with either a sense or an antisense probe. Signal was amplified by incubating the slide with rabbit anti-FITC/AP and detected by adding alkaline phosphatase substrate. Data showed that human resistin was expressed in bone marrow cells including messenchymal cells, but not in other tissues examined by this method. Human resistin expression in messenchymal cells was confirmed by in situ hybridization with cultured messenchymal cells.

### Example 4

### Taqman Analysis

To study the role of human resistin during adipocyte differentiation, human resistin expression during human adipocyte differentiation was detected by Taqman analysis using the following primers and probe set:

(SEQ ID NO: 10)

5′ AGCCATCAATGAGAGGATCCA-3′

(SEQ ID NO: 11)

5′ TCCAGGCCAATGCTGCTTAT-3′

(SEQ ID NO: 12)

5′ 6-FAM(TCGCCGGCTCCTAATATTTAGGGC)BHG-1 3′

Total RNAs isolated from cultured human pre-adipocyte and adipocyte were purchased from Zen-Bio. Lung total RNA as positive control was obtained from Clontech. These RNA preparations were treated with Dnase (Ambion) before first strand synthesis. The first strand synthesis was conducted by random priming using superscript first-strand synthesis system for RT-PCR (Invitrogen). Taqman analysis was performed in ABI PRISM, 7700 Sequence Detector.

Taqman analysis showed that low level human resistin nucleic acid was detected in pre-adipocyte RNA, but not in differentiated adipocyte RNA. This result is in agreement with Northern and in situ hybridization and suggests human resistin may play a role in adipocyte differentiation or in pre-adipocyte survival and proliferation.

### Example 5

### In Vitro Testing for Hematopietic Modulators

A. Human Megakaryocyte Assay

Resistin polypeptides can be assayed for ability to stimulate development of human megakaryocytes from CD34+ progenitor cells. CD34+ selected cells are obtained from bone marrow as described in Hokom, M. et al., Molecular Biology of Haematopoiesis 3:15, (1994) and are incubated in Iscove's modified Dulbecco's medium (IMDM; GIBCO) with 2 mM Glutamine, 2-mercaptoethanol (10⁻⁴ M), 1% bovine serum albumin, low density lipoprotein (40 μg/ml, Sigma); bovine pancreatic insulin (10 μg/ml), human transferrin (200 μg/ml), human recombinant thrombopoietin (50 ng/ml, R&D System); human recombinant stem cell factor (50 ng/ml, R&D Systems), human recombinant IL-3 (10 ng/ml, R&D System) and 1.1 mg/ml collagen and plus or minus various test concentrations resistin (100 ng/ml −1000 ng/ml). CD34+ cells are plated at 3300 cells/ml final concentrations on 2 well chamber slides. Cells are incubated at 37° C. for 12 days in 5% CO₂ in air, fixed to the culture wells with 1:3 methanol:acetone solution, and incubated with a monoclonal antibody, anti-GPIIb/IIIa, (StemCell Technologies). The immune reaction is developed with biotin conjugated goat anti-mouse IgG followed by avidin-alkaline phosphatase conjugate, identified by pink color, are counted with an inverted phase microscope at 100× magnification. Results are presented as the average number of megakaryocytes per well +/− standard error of the mean (SEM).

B. Activity of Resistin Polypeptides on Human Hematopoietic Progenitors

Human bone marrow CD34+ cells are purchased from Bio Whittaker and incubated in IMDM with 0.9% Methylcellulose, 2 mM Glutamine, 2-mercaptoethanol (104 M), 1% bovine serum albumin, bovine pancreatic insulin (10 μg/ml), human transferrin (200 μg/ml) and purified resistin at various concentrations (10-1500 ng/ml) and various combinations and concentrations of human stem cell factor (preferably 10-50 ng/ml), IL-3 (preferably 0.1-10 ng/ml), EPO (preferably 0.5-2.0 U/ml), and GM-CSF. CD34+ cells are plated at 1000 cells/ml final concentration in 35-mm dishes purchased from StemCell Technologies (Vancouver, Canada). Cells are incubated at 37° C. for 10-16 days in 5% CO₂ in air. Cells are fed as needed by replacing 400 μl of the medium with fresh medium. Colonies are scored under an inverted microscope. The cells are centrifuged and incubated 15 min. at 4° C. with 50 μg/ml human IgG (Sigma). Diagnostic antibodies, αCD 14-FITC for monocytes and αCD36-PE for erythroid cells are added for an additional 15 minutes. After a wash, cells are transferred to 12×75 mm tubes in a final volume of 1 ml containing 0.1 ml FlowCount Fluorosphere number (e.g. 5000). Analysis of data is accomplished by determining the number of cells of each lineage that are present in each well which is calculated based on the known number of fluorospheres. Total cells, monocytic erythroid and undefined lineages are determined. Data are subjected to statistical analysis using JMP 4 software. Unknowns are compared to negative controls using Dunnet's test at p=0.01. Accordingly, treatment with resistin polypeptide in the presence of 2 U/ml EPO and 50 ng/ml SCF may result in a significant increase in the size of erythroid colonies (BFU-E or GEMM). Treatment with EPO+IL3+SCF+resistin and treatment with EPO+SF+resistin increase CFU-GEMM numbers and BFU-E numbers compared to controls performed in the absence of resistin are shown below when the EPO concentration was 2U/ml, the IL-3 was 10 ng/ml and the SCF concentration was 50 ng/ml and resistin was at 200 ng/ml:

| Cytokine combination | CFU-GEMM per 1000 cells |
|---|---|
| 1. EPO + IL3 | 3 |
| 2. EPO + IL3 + resistin | 6 |

| | Primitive BFU-E |
|---|---|
| Cytokine combination | per 1000 cells |
| 1. EPO + SCF | 9 |
| 2. EPO + SCF + resistin | 18 |
| 3. EPO + SCF + IL-3 | 26 |
| 4. EPO + SCF + IL-3 + resistin | 38 |

C. Assay for Liquid Bone Marrow Culture

CD34+ human bone marrow cells are purchased from Poietic, BioWhittaker and are incubated in the following culture medium IMDM supplemented with 30% fetal bovine serum, antibiotics, 2 mM glutamine, 2-mercaptoethanol (10⁻⁴ M), and various combinations and concentration of resistin, human stem cell factor ((preferably 10 ng/ml), IL-3 (preferably 0.1 ng/ml), EPO (preferably 1 unit/ml) and/or GM-CSF (40 ng/ml). CD34+ cells were plated in polypropylene U-bottomed 96 well plates at 5000 cells/well and are cultured at 37° C., 5% CO₂ for 10 days with a breathable membrane to prevent evaporation. Feeding occurs at days 4 and 7 by replacing 80% of the medium with fresh medium. At day 10, the cells are transferred to V-bottomed plates and stained for CD41 (FITC) and CD36 (PE). Cells are then acquired on a flow cytometer in timed acquisition mode and compared to negative controls.

### Example 6

### In Vivo Testing for Hematopietic Modulators

A. Assays for Recovery of Blood Cells after Bone Marrow Transplantation.

Bone marrow is harvested by gently flushing the hind limbs of normal 8- to 10-week-old Balb/c mice (purchased from Harlan Sprague Dawley) using RPMI medium (GIBCO) containing 10% fetal calf serum (GIBCO). For some experiments, donor mice are pretreated with 5-fluorouracil (5-FU) at 150-mg/kg-body weight intraperitoneally (IP) 3 days before harvesting BM for infusion. After total body irradiation with 10.8 Gy (¹³⁷Cs at 126cGy/min, split dose with a minimum of 3 hours between doses), 1×10⁶ bone marrow cells are injected intravenously (IV) into sub-lethally irradiated mice. resistin (250 μg/kg body weight) are diluted in PBS and are injected subcutaneously in 0.2-mil volume daily starting on the same day as irradiation and infusion of donor bone marrow cells. Control mice receive the same volume of PBS. Administration of resistin polypeptides occurs in days 14-18. Mice are weighed every 2 to 4 days during the post-transplantation period. Hematologic analysis of leukocyte cell counts and platelet counts are performed on orbit bleeds on a CDC Mascot™ machine. Blood smears are stained with Wright-Giemsa using standard methods and examined at 100× for differentiation analysis. Peripheral blood hematocrits are performed by spinning capillary tubes for 5 minutes in a Model MB Micro-Capillary Centrifuge. Accordingly, resistin polypeptides may be used to accelerate recovery of peripheral blood cell counts.

B. Assays for Recovery of Blood Cells after Chemo-/Radiation Therapy

Eight- to ten-week old Balb/c mice (Harlan Sprague Dawley) are administered 5-fluorouracil (5-FU) at 150-mg/kg body weights IP 3 days before sub-lethal irradiation (0.6 Gy total body irradiation for 20-22 mg mouse). Resistin polypeptides (with or without EPO or EPO+SCF or EPO+SCF+IL3) are injected subcutaneously in 0.2 ml volumes daily starting on the same day as irradiation, optionally prior to irradiation. Negative control mice receive the same volume of PBS as the treated mice. Resistin polypeptide administration lasts for 14 days. The mice are analyzed at various days post-radiation. Mice are weighed every 2 to 4 days during the post-radiation period. Hematologic analysis of leukocyte cell counts and platelet counts are performed on orbit bleeds on a CDC Masco™ machine. Blood smears are stained with Wright-Giemsa using standard methods and examine at 100× for differentiation analysis. Peripheral blood hematocrits are performed by spinning capillary tubes for 5 minutes in a Model MB Micro-Capillary Centrifuge. Resistin polypeptides may be useful in accelerating recovery of peripheral blood cell counts after chemo-/radiation therapy.

C. Assays for Treatment of Anemia

Various animal models of anemia and hematopoietic disorder are known in the art and generally accepted as being indicative of the anemic condition. For instance, the exhypoxic polycythemic mouse bioassay may be used to quantify the incorporation of 5'Fe (iron) into newly synthesized red blood cells as a measure of the increase in erythropoiesis in mice in response to an exogenously administered test sample (e.g., a putative resistin agonist, resistin antagonist, resistin polynucleotide, resistin polypeptide, resistin fragment, resistin variant, and/or resistin antibody). The assay, as described in WO 0024893 (herein incorporated by reference), is a modification of the method of Cotes and Bangham (Nature 191:1065 (1961)).

The test agent(s) may be administered by any of several routes of administration (e.g. i.v., s.c., i.p., or by minipump or cannula) and suitable test animals include normal mice as well as resistin transgenic mice. Controls for non-specific effects for these treatments are done using vehicle with or without the active agent of similar composition in the same type animal monitoring the same parameters.

### Example 7

### Proliferation of Resistin Splenocytes in a Mixed Lymphocyte Reaction

In a mixed lymphocyte reaction assay, splenocytes from DBA/2 mice (Harlan Sprague Dawley, Indianapolis, Ind.) may be used as stimulator cells after being treated with mitomycin C. The responder cells are splenocytes isolated from C57B1/6 mice (Harlan Sprague Dawley) transplanted with the resistin gene or naïve mice. The suspensions of responder T-cells are cultured with allogeneic stimulator lymphocytes. The activating stimulus is the foreign histocompatibility antigen (usually MHC class I or class II molecules) expressed on the allogenic stimulator cells.

In brief, splenocytes from DBA/2 are added to 96-well plates at 1×10⁶ cells per well in RPMI+10% FBS and Pen/Strep. Splenocytes from either age matched C57B1/6 naïve mice or retroviral expressed resistin mice are added as responder cells to wells at either 0.5, 1, 2, 4, or 8×10⁵ cells per well. Control wells contained DBA stimulator splenocytes alone or C57B1/6 responder splenocytes alone. After 72 hours in vitro, wells are pulse labeled with 1 μCi of tritiated thymidine. After 18 hrs., cells are harvested and counted.

### Example 8

### Proliferation and Cytokine Secretion of Resistin Expressing Splenocytes Upon Antigenic Stimulation

Flat bottom 96 well plates are coated with 100 μl media (RPMI, 10% FBS) containing 5 μg/ml 1-CD3 per well. Plates are coated for 1.5 hrs. at 37° C., aspirated, and washed twice in PBS. Then, 4×10⁵ spleen cells in a 100 μl volume of media are added to each well and plates are incubated for 48 hrs., 37° C. After plates are centrifuged at 1200 rpm for 5 min., 100 μl of supernatant from each well is removed and transferred to 96-well U-bottom plates of which 10 μl is used for the cytokine secretion immunoassay according to standard procedures. Remainder of cells are pulse-labeled with 1 μCi of ³H-thymidine/well and incubated for another 24 hrs. prior to counting. In addition to activation of splenocytes by anti-CD3, other stimuli can be tested in the same manner. Preferred stimuli for testing include IL-2, ConA, PMA with ionomycin, and LPS.

### Example 9

### Exposure of Resistin Transgenic Mice to Sub-Lethal Doses of Radiation

Wild type and transgenic mice are irradiated at 600 cGy. The mice are analyzed at 3, 7, 10, 14, 21, 28 days post-radiation. Mice are weighed every 2 days during the post-radiation period. Hematologic analysis of leukocyte cell counts and platelet counts are performed on orbit bleeds on a CDC Mascot™ machine. Blood smears are stained with Wright-Giemsa using standard methods and examine at 100× for differentiation analysis. Peripheral blood hematocrits are performed by spinning capillary tubes for 5 minutes in a Model MB Micro-Capillary Centrifuge. Resistin may be used to accelerate the recovery of peripheral blood cell counts after exposure to sub-lethal doses of radiation.

### Example 10

### Resistin Increases the Numbers of CFU-GEMM in Mice

Mouse bone marrow cells are isolated from BDF-1 or C3H1 mice purchased from Jackson Laboratory. Cells from the femur marrow are cultured in methylcellulose or Agar culture medium (Stem Cell Technologies) in the presence of hemin. Colony growth is stimulated with the following combinations of recombinant growth factors with and without resistin (200 ng/ml): 1) Control Medium; 2) Epo (2 U/ml, R&D System); 3) Epo plus SCF (50 ng/ml, R&D System) and PWM-SCM (1%, Stem Cell Technologies); 4) GM-CSF (10 ng/ml); 5)GM-CSF plus SCF; 6) M-CSF(10 ng/ml); 7) M-CSF plus SCF. After culture at 37° C. for 1 week, different colonies are counted from each dish under an inverted microscope. Group mean and SD are calculated. Resistin may significantly increase the numbers of mouse CFU-GEMM (colony-forming unit-granulocyte, erythrocyte, megakaryocyte and monocyte).

### Example 11

### CFU-GEMM Levels in Resistin Transgenic Mice

Mouse bone marrow cells and splenocytes are isolated from resistin transgenic mice and age-matched wild type mice. Then, 1×10⁵ mononuclear cells from the bone marrow or 1×10⁶ mononuclear cells from spleen were cultured in methylcellulose or Agar culture medium (Stem Cell Technologies) in the presence of hemin. Colony growth is stimulated with the following combinations of recombinant growth factors with and without resistin (200 ng/ml): 1) Control Medium; 2) EPO (2 U/ml, R&D System); 3) EPO plus SCF (SOng/ml, R&D System) and PWM-SCM (1%, Stem Cell Technologies); 4) GM-CSF (10 ng/ml); 5) GM-CSF plus SCF; 6) M-CSF (10 ng/ml). After culture at 37° C. for 1 week, different colonies are counted from each dish under an inverted microscope. Group mean and SD are calculated. Resistin transgenic mice may have greater numbers of CFU-GEMM in both bone marrow and spleen.

### Example 12

### Administration of Resistin to Mice Increases the CFU-GEMM numbers

Eight-to-ten-week-old female splenectomized BDF1 mice (Harlan) are used in the studies. All mice are earpunched for identification. Groups are:
1. diluent (PBS+0.5% homologous mouse serum)
2. Resistin in diluent (5 μg per injection)
3. EPO in diluent (10 units per injection)
4. Resistin+EPO in diluent (same amounts as above)
5. SCF (1.5 μg/injection)+EPO (10 units/injection) in diluent
6. Resistin+EPO+SCF in diluent (same amounts as above)

The bone marrow cells from splenectomized BDF1 mice that are injected with resistin alone or with various combinations of cytokines plus or minus resistin for 11 days are then cultured in methylcellulose-based medium (Stem Cell Technologies, Vancouver, BC) in the presence of hemin. Colony growth is stimulated with the following combinations of recombinant growth factors Epo (2 U/ml, R&D System), SCF (SOng/ml, R&D System) and IL-3(R&D System). The colonies of erytiroid, CFU-GM and CFU-GEMM cells are scored under microscope. The total number of each kind of progenitors in one femur is calculated and the data statistically analyzed. Resistin, alone or in combination with other cytokines, may significantly increase numbers of mouse CFU-GEMM in vivo.

### REFERENCES

1. Banerjee, R. and M. Lazar. J. Biol. Chem. 276:25970 (2001).
2. Cao, H. and R. Hegele. J. Human Genet. 46:553 (2001).
3. Fasshauer, M., et al. FEBS Letters 500:60 (2001).
4. Fasshauer, M., et al. Biochem. Biophys. Res. Com. 288:1027 (2001).
5. Flier, J. S. Nature 409:292 (2001).
6. Hartman, H., et al. J. Biol. Chem. In press. (2002).
7. Janke, J., et al. Obesity Research. 10:1 (2002).
8. Juan, C-C., et al. Biochem. Biophys. Res. Corn. 289:1328 (2001).
9. Kim, K., et al. J. Biol. Chem. 276:11252 (2001).
10. McTernan, C., et al. Lancet 359:46 (2002).
11. Nagaev, I and U. Smith. Biochem. and Biophys. Res. Com. 285:561 (2001).
12. Osawa, H., et al. Diabetes. 51:863 (2002).
13. Savage, D., et al. Diabetes 40:2199 (2001).
14. Sentinelli, F., et al. Diabetes 51:860 (2002).
15. Shuldiner, A., et al. New Eng. J. Med. 345:1345 (2001).
16. Smith, U. Obesity Research. 10:61 (2002).
17. Steppan, C., et al. Nature 409:307 (2001).
18. Steppan, C., et al. Proc. Natl. Acad. Sci. 98:502 (2001).
19. Steppan, C. and M. Lazar. Trends Endoc. & Met. 13:18 (2002).
20. Way, J., et al. J. Biol. Chem. 276:25651 (2001).

1. A method of increasing the number of one or more type(s) of hematopoietic progenitor cells in a mammal in need thereof comprising administering a therapeutically effective amount of a resistin polypeptide.

2. The method of claim 1 wherein the type of hematopoietic progenitor cells is selected from the group consisting of CFU-GEMM or BFU-E.

3. The method of claim 1 wherein the one or more types of hematopoietic progenitor cells includes CFU-GEMM.

4. A method of increasing hematocrit in a mammal in need thereof comprising administering a therapeutically effective amount of a resistin polypeptide.

5. A method for treating or preventing a hematopoietic disorder in a mammal comprising the administration to said mammal in need of such treatment a pharmaceutical composition comprising a therapeutically effective amount of a resistin polypeptide.

6. A method of treating or preventing anemia, thrombocytopenia and/or neutropenia in a mammal comprising the administration to said mammal in need of such treatment a pharmaceutical composition comprising a therapeutically effective amount of a resistin polypeptide.

7. A method of treating or preventing leukemia or lymphomas in a mammal comprising the administration to said mammal in need of such treatment a pharmaceutical composition comprising a therapeutically effective amount of a resistin polypeptide.

8. A method of treating a solid tumor of non-hematopoietic origin in a mammal comprising the administration to said mammal in need of such treatment a pharmaceutical composition comprising a therapeutically effective amount of a resistin polypeptide.

9. A method of treating an autoimmune condition in a mammal comprising the administration to said mammal in need of such treatment a pharmaceutical composition comprising a therapeutically effective amount of a resistin polypeptide.

10. The method of any one of claims 1-9 further comprising administering a therapeutically effective amount of at least one hematopoietic cytokine in addition to the resistin polypeptide.

11. The method of any one of claims 1-10 wherein said resistin polypeptide is selected from the group consisting of:
a) a polypeptide comprising residues 1-108 of SEQ ID NO: 2;
b) a polypeptide comprising residues from about 18-108 of SEQ ID NO: 2;
c) a polypeptide comprising residues 1-108 of SEQ ID NO: 2 with 1, 2, 3, 4 or 5 amino acid substitutions, insertions or deletions and which retains substantially similar activity to the polypeptide comprising residues 1-108 of SEQ ID NO:2; and
d) a polypeptide comprising residues from about 18-108 of SEQ ID NO: 2 with 1, 2, 3, 4 or 5 amino acid substitutions, insertions or deletions and which retains substantially similar activity to the polypeptide comprising residues 1-108 of SEQ ID NO:2.

12. A pharmaceutical composition comprising a hematopoietic progenitor cell stimulating amount of a resistin polypeptide and a pharmaceutically acceptable carrier, diluent or excipient.

13. A pharmaceutical composition comprising a CFU-GEMM stimulating amount of a resistin polypeptide and a pharmaceutically acceptable carrier, diluent or excipient.

14. The pharmaceutical composition as in claims 12 or 13 further comprising at least one hematopoietic cytokine in addition to the resistin polypeptide.

15. The pharmaceutical composition of any one of claims 12-14 wherein said resistin polypeptide is selected from the group consisting of:
a) a polypeptide comprising residues 1-108 of SEQ ID NO: 2;
b) a polypeptide comprising residues from about 18-108 of SEQ ID NO: 2;
c) a polypeptide comprising residues 1-108 of SEQ ID NO: 2 with 1, 2, 3, 4 or 5 amino acid substitutions, insertions or deletions and which retains substantially similar activity to the polypeptide comprising residues 1-108 of SEQ ID NO:2; and
d) a polypeptide comprising residues from about 18-108 of SEQ ID NO: 2 with 1, 2, 3, 4 or 5 amino acid substitutions, insertions or deletions and which retains substantially similar activity to the polypeptide comprising residues 1-108 of SEQ ID NO:2.


SEQUENCE LISTING (14 sequences)

SEQ ID NO: 1 - DNA, 327 bp, Homo sapiens
  misc_feature (1)..(327): Human Resistin Polynucleotide
atgaaagctctctgtctcctcctcctccctgtcctggggctgttggtgtctagcaagaccctgtgctccatggaagaagccatcaatgagaggatccaggaggtcgccggctccctaatatttagggcaataagcagcattggcctggagtgccagagcgtcacctccaggggggacctggctacttgcccccgaggcttcgccgtcaccggctgcacttgtggctccgcctgtggctcgtgggatgtgcgcgccgagaccacatgtcactgccagtgcgcgggcatggactggaccggagcgcgctgctgtcgtgtgcagccctga

SEQ ID NO: 2 - protein, 108 aa, Homo sapiens
  MISC_FEATURE (1)..(108): Human Resistin Polypeptide
MKALCLLLLPVLGLLVSSKTLCSMEEAINERIQEVAGSLIFRAISSIGLECQSVTSRGDLATCPRGFAVTGCTCGSACGSWDVRAETTCHCQCAGMDWTGARCCRVQP

SEQ ID NO: 3 - protein, 90 aa, Homo sapiens
  MISC_FEATURE (1)..(90): Mature Human Resistin Polypeptide
KTLCSMEEAINERIQEVAGSLIFRAISSIGLECQSVTSRGDLATCPRGFAVTGCTCGSACGSWDVRAETTCHCQCAGMDWTGARCCRVQP

SEQ ID NO: 4 - protein, 108 aa, Homo sapiens; identical to SEQ ID NO: 2
  MISC_FEATURE (1)..(108): Human Resistin Allelic Variant

SEQ ID NO: 5 - protein, 108 aa, Homo sapiens; identical to SEQ ID NO: 2
  MISC_FEATURE (1)..(108): Human Resistin Allelic Variant

SEQ ID NO: 6 - protein, 108 aa, Homo sapiens
  MISC_FEATURE (1)..(108): Human Resistin Allelic Variant
MKALCLLLLPVLGLLVSSKTLCSMEEAINERIQEVAGSLIFRAISSIGRGSESVTSRGDLATCPRGFAVTGCTCGSACGSWDVRAETTCHCQCAGMDWTGARCCRVQP

SEQ ID NO: 7 - protein, 107 aa, Homo sapiens
  MISC_FEATURE (1)..(107): Human Resistin Allelic Variant
MKALCLLLLPVLGLLVSSKTLCSMEEAINERQEVAGSLIFRAISSIGXXXXSVTSRGDLATCPRGFAVTGCTCGSACGSWDVRAETTCHCQCAGMDWTGARCCRVQP

SEQ ID NO: 8 - DNA, 39 bp, Artificial Sequence
  Synthetic Construct
gatcggcgcgccagccaccatgaaagctctctgtctcct

SEQ ID NO: 9 - DNA, 29 bp, Artificial Sequence
  Synthetic Construct
cgcgatatcgggctgcacacgacagcagc

SEQ ID NO: 10 - DNA, 21 bp, Artificial Sequence
  Synthetic Construct
agccatcaatgagaggatcca

SEQ ID NO: 11 - DNA, 20 bp, Artificial Sequence
  Synthetic Construct
tccaggccaatgctgcttat

SEQ ID NO: 12 - DNA, 24 bp, Artificial Sequence
  Synthetic Construct
tcgccggctcctaatatttagggc

SEQ ID NO: 13 - protein, 114 aa, rattus sp.
  MISC_FEATURE (1)..(114): Rat resistin protein
MKNLSFLLLFLFFLVLGLLGPSMSLCPMDEAISKKINQDFSSLLPAAMKNTVLHCWSVSSRGRLASCPEGTTVTSCSCGSGCGSWDVREDTMCHCQCGSIDWTAARCCTLRVGS

SEQ ID NO: 14 - protein, 114 aa, mus sp.
  MISC_FEATURE (1)..(114): Mouse resistin protein
MKNLSFPLLFLFFLVPELLGSSMPLCPIDEAIDKKIKQDFNSLFPNAIKNIGLNCWTVSSRGKLASCPEGTAVLSCSCGSACGSWDIREEKVCHCQCARIDWTAARCCKLQVAS